(19) United States
(12) Patent Application Publication
(10) Pub. No.: US 2004/0058320 A1
(43) Pub. Date: Mar. 25, 2004

(54) Reagents and methods for identifying and modulating expression of tumor senescence genes

(76) Inventors: Igor B. Roninson, Wilmette, IL (US); Bey-Dih Chang, Lombard, IL (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/032,264

(22) Filed: Dec. 21, 2001

Related U.S. Application Data
(63) Continuation of application No. 60/257,907, filed on Dec. 21, 2000.
(60) Provisional application No. 60/341,425, filed on Dec. 17, 2001.

(51) Int. Cl. C12Q 1/68; G01N 33/574

(52) U.S. Cl. 435/6; 435/7.23

(57) ABSTRACT

This invention identifies tumor senescence genes induced by treatment with cytotoxic agents. The invention provides reagents and methods for identifying compounds that induce expression of these cellular genes and produce cellular senescence, particularly senescence in tumor cells. The invention also provides reagents that are recombinant mammalian cells containing recombinant expression constructs that express a reporter gene under the transcriptional control of a promoter for a gene the expression of which is modulated in senescent cells, and methods for using such cells to identify compounds that modulate expression of these cellular genes.

This application claims priority to U.S. Provisional Application Serial No. 60/257,907, filed Dec. 21, 2000 and U.S. Provisional Application Serial No. 60/______, filed Dec. 17, 2001.

This application was supported by a grant from the National Institutes of Health, No. ______. The government may have certain rights in this invention.

### BACKGROUND OF THE INVENTION

1. Field of the Invention

This invention is related to changes in cellular gene expression and compounds that produce changes in cellular gene expression. In particular, the invention is related to the identification of genes the expression of which is associated with the development of senescence in mammalian tumor cells upon treatment with cytotoxic agents, including chemotherapeutic drugs, such as doxorubicin, and ionizing radiation. More specifically, the invention provides methods for identifying compounds that modulate expression of these cellular genes. The invention also provides reagents that are recombinant mammalian cells containing recombinant expression constructs that express a reporter gene under the transcriptional control of a promoter for a senescence-associated gene expression, and methods for using such cells for identifying compounds that modulate expression of these cellular genes and produce senescence in said cells. Compounds identified using the methods of the invention are provided for use in therapeutic methods for treating diseases and disorders relating to abnormal cellular proliferation or neoplastic cell growth. Diagnostic methods, particularly methods for monitoring the efficacy of anticancer treatment regimes, are also provided by this invention.

2. Summary of the Related Art

Cancer remains one of the leading causes of death in the United States. Current treatment for cancer includes chemotherapy and radiation, but these treatments are not invariably cytotoxic to all tumor cells. Some of the cells that survive treatment recover and resume proliferation, while others undergo permanent growth arrest. Irreversible proliferation arrest in tumor cells treated with anticancer agents may result from cell death or permanent growth arrest. Although the mechanism of damage-induced cell death is a subject of active investigation, little is known about the determinants of terminal growth arrest in tumor cells.

Exposure of different tumor cell lines to various anticancer agents in vitro and in vivo induces long-term growth arrest with phenotypic features of cell senescence, such as cell enlargement, increased adhesion and granularity, and senescence-associated β-galactosidase activity (SA-β-ga1; Chang et al., 1999a, Cancer Res. 59: 3761-3767). Induction of the senescent phenotype in treated tumor cells has been observed in cells treated with a variety of cytotoxic agents, such as doxorubicin, aphidicolin, cisplatin, ionizing radiation, cytarabine, etoposide or taxol; this response is detectable in treated tumor cells even at the lowest concentration of a cytotoxic agent that produces detectable growth inhibition (Chang et al., 1999a, ibid.). Senescence of tumor cells can be produced upon treatment not only with cytotoxic agents but also with vitamin A derivatives, retinoids, under conditions that produce growth inhibition with only minimal cytotoxicity (Chang et al., 1999a, ibid.). Retinoid-induced senescence in breast carcinoma cells is associated with co-induction of several growth-inhibitory genes, as described in Dokmanovic et al. (2002, Cancer Biol. Ther. 1: 16-19) and in co-owned and co-pending U.S. Ser. No. 09/865,879, filed May 25, 2001, incorporated by reference herein. Tumor cells can also be induced to undergo senescence through ectopic expression of tumor suppressors (Sugrue et al., 1997, Proc. Natl. Acad. Sci. USA 94: 9648-9653; Uhrbom et al., 1997, Oncogene 15: 505-514; Xu et al., 1997, Oncogene 15: 2589-2596) or oncogene inhibition. For example, inhibition of papillomavirus oncoproteins E6 and E7 in cervical carcinoma cell lines was found to induce senescence-like growth arrest in almost 100% of cells (Goodwin, 2000, Proc. Natl. Acad. Sci. USA 97: 10978-10983). Activation of the senescence program in tumor cells appears therefore to be a feasible biological approach to cancer therapy.

There remains a need in the art to identify genes that are induced when a cell, particularly a tumor cell, becomes senescent, both as markers for the senescence phenotype and as targets for inducing senescence in said cells. There is also a need in the art to identify cells, particularly tumor cells that have become senescent in response to treatment, particularly anticancer treatment, to assess the efficacy of such treatment. There is further a need in the art to identify compounds that induce senescence in mammalian cells, particularly tumor cells, as a way to improve treatment of proliferative disorders such as cancer.

### SUMMARY OF THE INVENTION

This invention provides genes that are induced or repressed in senescent cells and arise upon treatment with cytotoxic agents, as well as reagents and methods for identifying compounds that induce or repress such genes. The invention also advantageously provides compounds that mimic the effects of cytotoxic agents in inhibiting the growth of tumor cells without producing toxicity associated with these agents. Most preferably the mimicked effect is induction of senescence in mammalian tumor cells.

In a first aspect, the invention provides a method for identifying a compound that induces senescence in a mammalian cell. In one embodiment, the method comprises the steps of culturing the mammalian cell in the presence and absence of the compound; assaying expression of at least one cellular gene set forth in Table 2A in said cell in the presence of the compound with expression of said gene in the cell in the absence of the compound; and identifying compounds that induce senescence when expression of at least one cellular gene in Table 2A is higher in the presence of the compound than in the absence of the compound. In a preferred embodiment, the mammalian cell is a p53 deficient cell. In other preferred embodiments, the mammalian cell is a tumor cell. Preferably, the cellular gene is a human gene, most preferably BTG1, BTG2, EPLIN, WIP1, Maspin, MIC-1, IGFBP-6 or amphiregulin. Expression of cellular genes according to the method is preferably detected by hybridization to a complementary nucleic acid, by using an immunological reagent or by assaying for an activity of the cellular gene product.

In alternative embodiments, the mammalian cell is a recombinant mammalian cell comprising a reporter gene operably linked to a promoter from a cellular gene in Table 2A. In these embodiments, induction of at least one of the cellular genes in Table 2A is assayed using the recombinant mammalian cell and increased expression of the reporter gene detected in the presence and absence of the compound. In further preferred embodiments, the method comprises the additional steps of assaying the mammalian cell in the presence and absence of the test compound for expression of one or more genes in Table 2B; and identifying compounds wherein expression of the genes in Table 2B is not greater in the presence of the compound than in the absence of the compound. Expression of reporter genes according to the method is preferably detected by hybridization to a complementary nucleic acid, by using an immunological reagent or by assaying for an activity of the reporter gene product.

In additional embodiments of the first aspect of the invention, the method for identifying a compound that induces senescence in a mammalian cell comprises the steps of culturing the mammalian cell in the presence and absence of the compound; assaying expression of at least one cellular gene set forth in Table 2A in said cell in the presence of the compound with expression of said gene in the cell in the absence of the compound; assaying the recombinant mammalian cell for cell growth and morphological features of senescence; and identifying compounds that induce senescence when expression of at least one cellular gene in Table 2A is higher in the presence of the compound than in the absence of the compound and the cells are growth-inhibited and express morphological features of senescence in the presence of the compound. In a preferred embodiment, the mammalian cell is a p53 deficient cell. In other preferred embodiments, the mammalian cell is a tumor cell. Preferably, the cellular gene is a human gene, most preferably BTG1, BTG2, EPLIN, WIP1, Maspin, MIC-1, IGFBP-6 or amphiregulin. Expression of cellular genes according to the method is preferably detected by hybridization to a complementary nucleic acid, by using an immunological reagent or by assaying for an activity of the cellular gene product.

In alternative embodiments, the mammalian cell is a recombinant mammalian cell comprising a reporter gene operably linked to a promoter from a cellular gene in Table 2A. In these embodiments, induction of at least one of the cellular genes in Table 2A is assayed using the recombinant mammalian cell and increased expression of the reporter gene detected in the presence and absence of the compound. In further preferred embodiments, the method comprises the additional steps of assaying the mammalian cell in the presence and absence of the test compound for expression of one or more genes in Table 2B; and identifying compounds wherein expression of the genes in Table 2B is not greater in the presence of the compound than in the absence of the compound. Expression of reporter genes according to the method is preferably detected by hybridization to a complementary nucleic acid, by using an immunological reagent or by assaying for an activity of the reporter gene product.

In a second aspect, the invention provides a method for identifying a compound that induces senescence in a mammalian cell. In one embodiment, the method comprises the steps of culturing the mammalian cell in the presence and absence of the compound; assaying expression of at least one cellular gene set forth in Table 1 in said cell in the presence of the compound with expression of said gene in the cell in the absence of the compound; and identifying compounds that induce senescence when expression of at least one cellular gene in Table 1 is lower in the presence of the compound than in the absence of the compound. In a preferred embodiment, the mammalian cell is a p53 deficient cell. In other preferred embodiments, the mammalian cell is a tumor cell. Preferably, the cellular gene is a human gene, most preferably HFH-11, STEAP, RHAMM, INSIG1, LRPR1. Expression of cellular genes according to the method is preferably detected by hybridization to a complementary nucleic acid, by using an immunological reagent or by assaying for an activity of the cellular gene product.

In alternative embodiments, the mammalian cell is a recombinant mammalian cell comprising a reporter gene operably linked to a promoter from a cellular gene in Table 1. In these embodiments, induction of at least one of the cellular genes in Table 1 is assayed using the recombinant mammalian cell and decreased expression of the reporter gene detected in the presence and absence of the compound. In further preferred embodiments, the method comprises the additional steps of assaying the mammalian cell in the presence and absence of the test compound for expression of one or more genes in Table 2B; and identifying compounds wherein expression of the genes in Table 2B is not greater in the presence of the compound than in the absence of the compound. Expression of reporter genes according to the method is preferably detected by hybridization to a complementary nucleic acid, by using an immunological reagent or by assaying for an activity of the reporter gene product.

In additional embodiments of the second aspect of the invention, the method for identifying a compound that induces senescence in a mammalian cell comprises the steps of culturing the mammalian cell in the presence and absence of the compound; assaying expression of at least one cellular gene set forth in Table 1 in said cell in the presence of the compound with expression of said gene in the cell in the absence of the compound; assaying the recombinant mammalian cell for cell growth and morphological features of senescence; and identifying compounds that induce senescence when expression of at least one cellular gene in Table 1 is lower in the presence of the compound than in the absence of the compound and the cells are growth-inhibited and express morphological features of senescence in the presence of the compound. In a preferred embodiment, the mammalian cell is a p53 deficient cell. In other preferred embodiments, the mammalian cell is a tumor cell. Preferably, the cellular gene is a human gene, most preferably HFH-11, STEAP, RHAMM, INSIG1, LRPR1. Expression of cellular genes according to the method is preferably detected by hybridization to a complementary nucleic acid, by using an immunological reagent or by assaying for an activity of the cellular gene product.

In alternative embodiments, the mammalian cell is a recombinant mammalian cell comprising a reporter gene operably linked to a promoter from a cellular gene in Table 1. In these embodiments, inhibition of at least one of the cellular genes in Table 1 is assayed using the recombinant mammalian cell and decreased expression of the reporter gene detected in the presence and absence of the compound. In further preferred embodiments, the method comprises the additional steps of assaying the mammalian cell in the presence and absence of the test compound for expression of one or more genes in Table 2B; and identifying compounds wherein expression of the genes in Table 2B is not greater in the presence of the compound than in the absence of the compound. Expression of reporter genes according to the method is preferably detected by hybridization to a complementary nucleic acid, by using an immunological reagent or by assaying for an activity of the reporter gene product.

In a third aspect, the invention provides compounds produced according to the methods of the invention, most preferably embodiments of the methods of the invention whereby the method comprises the additional steps of assaying the mammalian cell in the presence and absence of the test compound for expression of one or more genes in Table 2B; and identifying compounds wherein expression of the genes in Table 2B is not greater in the presence of the compound than in the absence of the compound.

The invention in a fourth aspect provides a method for assessing efficacy of a treatment of a disease or condition relating to abnormal cell proliferation or neoplastic cell growth. The method comprises the steps of: obtaining a biological sample comprising cells from an animal having a disease or condition relating to abnormal cell proliferation or neoplastic cell growth before treatment and after treatment; comparing expression of at least one gene in Table 1, 2A or 2B after treatment with expression of said genes before treatment; and determining that said treatment has efficacy for treating the disease or condition relating to abnormal cell proliferation or neoplastic cell growth if expression of at least one gene in Table 2A and 2B is higher after treatment than before treatment or expression of at least one gene in Table 1 is lower after treatment than before treatment. In preferred embodiments, the biological sample comprises tumor cells. Preferably, the gene is a cellular gene in Table 2A, most preferably wherein at least one cellular gene is a human gene that is BTG1, BTG2, EPLIN, WIP1, Maspin, MIC-1, IGFBP-6 or amphiregulin. In alternative preferred embodiments, the gene is a cellular gene in Table 1, most preferably a human gene that is HFH-11, STEAP, RHAMM, INSIG1, and LRPR1. Expression of cellular genes according to the method is preferably detected by hybridization to a complementary nucleic acid, by using an immunological reagent or by assaying for an activity of the cellular gene product.

In a fifth aspect, the invention provides a method for treating a disease or condition relating to abnormal cell proliferation or neoplastic cell growth, most preferably cancer. The method of the invention comprises the steps of administering to an animal having said disease or condition a therapeutically effective amount of a compound produced according to the inventive methods of the invention, most preferably embodiments of the methods of the invention whereby the method comprises the additional steps of assaying the mammalian cell in the presence and absence of the test compound for expression of one or more genes in Table 2B; and identifying compounds wherein expression of the genes in Table 2B is not greater in the presence of the compound than in the absence of the compound.

In a sixth aspect, the invention provides methods for identifying a compound that inhibits senescence-associated induction of cellular gene expression. In preferred embodiments of this aspect, the method comprises the steps of contacting the cell with a cytotoxic agent at a concentration of said agent that inhibits cell growth; assaying the cell in the presence and absence of the compound for changes in expression of cellular genes induced when cells become senescent; and identifying the compound as an inhibitor of senescence-associated induction of cellular gene expression if expression of the above cellular genes is induced in the absence of the compound but is not induced in the presence of the compound. In preferred embodiments, the cellular gene is a human gene that is cyclin D1, serum-inducible kinase, CYR61, prosaposin, transforming growth factor α (TGFα), kallikrein 7, calpain-L2, neurosin, plasminogen activator, urokinase, amyloid beta (A4) precursor protein (βAPP), or integral membrane protein 2B (BRI/ITM2B). In a preferred embodiment, the mammalian cell is a p53 deficient cell. In other preferred embodiments, the mammalian cell is a tumor cell. Expression of cellular genes according to the method is preferably detected by hybridization to a complementary nucleic acid, by using an immunological reagent or by assaying for an activity of the cellular gene product.

In alternative embodiments, the mammalian cell is a recombinant mammalian cell comprising a reporter gene operably linked to a promoter from a human gene that is cyclin D1, serum-inducible kinase, CYR61, prosaposin, transforming growth factor α □(TGFα), kallikrein 7, calpain-L2, neurosin, plasminogen activator, urokinase, amyloid beta (A4) precursor protein (βAPP), or integral membrane protein 2B (BRI/ITM2B). In these embodiments, the method comprises the steps of contacting the mammalian cell with a cytotoxic agent at a concentration of said agent that inhibits cell growth; assaying the mammalian cell in the presence and absence of the test compound for expression of the reporter gene; and identifying compounds wherein expression of the reporter gene is not greater in the presence of the compound than in the absence of the compound. Expression of the reporter gene according to the method is preferably detected by hybridization to a complementary nucleic acid, by using an immunological reagent or by assaying for an activity of the reporter gene product.

The invention also provides methods for monitoring the efficacy of treatment. In these embodiments, tumor cells that have become senescent and are no longer able to grow are identified and distinguished from tumor cells that recover and proliferate after treatment. Senescence marker detection in biopsy samples from tumors obtained after patient treatment is used as an indicator of treatment response.

Specific preferred embodiments of the present invention will become evident from the following more detailed description of certain preferred embodiments and the claims.

### BRIEF DESCRIPTION OF THE DRAWINGS

FIG. 1A is a fluorescence-activated cell sorting (FACS) profile of proliferating and growth-arrested fractions of doxorubicin-treated HCT116 cells. Cells are sorted based on PKH2 fluorescence on the indicated days after release from doxorubicin. PKH2^{lo} population of proliferating cells appears on day 4 and separates from the PKH2^{hi} (growth-arrested) population by day 6.

FIG. 1B is a FACS profile of proliferating and growth-arrested fractions of doxorubicin-treated HCT116 cells separated on the basis of DNA content. Exponentially growing HCT116 cells have a peak at G1, while the PKH2^{hi} population isolated 9 days after drug treatment has a peak at G2/M.

FIG. 1C is a photograph showing SA-β-ga1 staining of PKH2^{hi} and PKH2^{lo} populations, separated 6 days after release from the drug. Both panels are photographed at the same 200× magnification.

FIG. 1D is a photograph showing colony formation by PKH2^{hi} and PKH2^{lo} populations, separated 9 days after drug treatment and plated at 10,000 live (PI-negative) cells per 10-cm plate.

FIGS. 2A and 2B are photographs of RT-PCR analysis of changes in the expression of the indicated senescence-associated genes. β-actin was used as a normalization standard. FIG. 2A shows a comparison of gene expression in proliferating (PKH2^{lo}) and senescent (PKH₂^{hi}) populations of HCT116 cells, separated 9 days after doxorubicin treatment. FIG. 2B is a comparison of gene expression in the unsorted populations of wild-type, p2−/− and p53−/− HCT116 cells, before and after 24-hr treatment with 200 nM doxorubicin, and on the indicated days after release from the drug. Genes were designated as p53− or p21-dependent if changes in their expression became detectable at a later day or were less pronounced in the p53−/− orp21−/− lines than in the wild-type cells.

FIGS. 3A and 3B are photographs of immunoblotting analysis of changes in p53 and the indicated protein products of genes that show altered expression in drug-induced senescence. M-actin was used as a normalization standard. FIG. 3A shows the results of immunoblotting of wild type HCT116 cells that were either untreated, treated for two days with 200 nM doxorubicin, or sorted into proliferating (PKH2^{lo}) and senescent (PKH2^{hi} ) cell populations 9 days after doxorubicin treatment. FIG. 3B shows the p53 dependence of p21 induction in doxorubicin-treated HCT116 cells, through immunoblotting analysis of the wild type, p21−/− and p53−/− HCT116 cell lines treated with doxorubicin for the indicated number of days.

### DETAILED DESCRIPTION OF THE PREFERRED EMBODIMENTS

This invention provides genes the expression of which is modulated in cells that become senescent upon treatment with cytotoxic agents. The invention also provides methods for identifying compounds that mimic the gene expression modulating properties of cytotoxic agents but lack toxicity that is characteristic of chemotherapeutic drug treatment, as well as the compounds identified by the methods. Diagnostic and therapeutic treatment methods are provided as set forth more particularly herein.

For the purposes of this invention, the term “senescence” will be understood to include permanent cessation of DNA replication and cell growth not reversible by growth factors, such as occurs at the end of the proliferative lifespan of normal cells or in normal or tumor cells in response to cytotoxic drugs, DNA damage or other cellular insult. Senescence is also characterized by certain morphological features, including increased size, flattened morphology increased granularity, and senescence-associated β-galactosidase activity (SA-β-gal).

As used herein, the term “senescence-associated gene” is intended to encompass genes the expression of which is modulated (either induced or repressed) when a cell expresses a senescent phenotype, particularly a senescence phenotype produced by contacting the cells with a cytotoxic agent. Most preferably, the term will be understood to refer to the genes disclosed herein, inter alia, in Tables 1 and 2.

Senescence can be conveniently induced in mammalian cells by contacting the cells with a dose of a cytotoxic agent that inhibits cell growth (as disclosed in Chang et al., 1999a, ibid.). Cell growth is determined by comparing the number of cells cultured in the presence and absence of the agent and detecting growth inhibition when there are fewer cells in the presence of the agent than in the absence of the agent after an equivalent culture period of time Examples of such cytotoxic agents include but are not limited to doxorubicin, aphidicolin, cisplatin, cytarabine, etoposide, taxol and ionizing radiation. Appropriate dosages will vary with different cell types; the determination of the dose that induces senescence is within the skill of one having ordinary skill in the art, as disclosed in Chang et al., 1999a, ibid.

For the purposes of this invention, reference to “a cell” or “cells” is intended to be equivalent, and particularly encompasses in vitro cultures of mammalian cells grown and maintained as known in the art, as well as biological samples obtained, inter alia, from tumor specimens in vivo.

For the purposes of this invention, reference to “cellular genes” in the plural is intended to encompass a single gene as well as two or more genes. It will also be understood by those with skill in the art that effects of modulation of cellular gene expression, or reporter constructs under the transcriptional control of promoters derived from cellular genes, can be detected in a first gene and then the effect replicated by testing a second or any number of additional genes or reporter gene constructs. Alternatively, expression of two or more genes or reporter gene constructs can be assayed simultaneously within the scope of this invention.

The methods of the invention can be practiced using any mammalian cell, preferably a rodent or primate cell, most preferably a human cell that can develop a senescence phenotype in response to a cytotoxic agent. Preferred cells include mammalian cells, preferably rodent or primate cells, and most preferably human cells. In certain embodiments, most preferred cells are p53 deficient cells, that are cells expressing less than the normal amount or less than the normal functional activity of tumor suppressor p53 as the result of mutation, deletion, recombination, chromosome loss or genetic manipulation.

In certain embodiments, the methods of the invention are advantageously practiced using recombinant mammalian cells comprising a recombinant expression construct encoding a reporter gene operably linked to a promoter from a gene that is induced in senescent cells. Preferred reporter genes comprising said constructs include firefly luciferase, chloramphenicol acetyltransferase, beta-galactosidase, green fluorescent protein (GFP), alkaline phosphatase and most particularly a commercially-available GFP-luciferase fusion gene. Most preferred promoters comprising the recombinant expression constructs of the invention are promoters from a cellular gene known to be induced in senescent cells. The cellular gene promoter is advantageously from a gene identified in Table 2A herein. In more preferred embodiments, the cellular promoter is from BTG1, BTG2, EPLIN, WIP1, Maspin, MIC-1, IGFBP-6 or amphiregulin. In alternative embodiments, the cellular gene promoter is from a gene that is repressed in senescent cells. Preferred promoters of this type include promoters is from a gene identified in Table 1 herein. In more preferred embodiments, the cellular promoter is from HFH-11, STEAP, RHAMM, INSIG1, LRPR1.

Promoter sequences from some of these genes are known in the art. These include: cyclin D1 (Motokura & Arnold, 1993, Genes Chromosomes Cancer 7: 89-95); CYR61 (Latinkic et al., 1991, Nucleic Acids Res. 19: 3261-7); prosaposin (Sun et al., 1998, Gene 218: 23-34); transforming growth factor a (TGFα; Raja et al., 1991, Mol. Endocrinol. 5: 514-20); kallikrein 7 (Yousef et al., 2000, Gene 254: 119-128); calpain-L2 (Suzuki et al., 1995, Biol Chem Hoppe Seyler. 376: 523-9); plasminogen activator urokinase (Riccio et al., 1985, Nucleic Acids Res. 13: 2759-71); and amyloid beta (A4) precursor protein (βAPP; Lahiri & Robakis, 1991, Brain Res. Molec. Brain Res. 9: 253-257).

For other genes, promoter sequences can be readily isolated from a region of genomic DNA within about 5 kilobases (and more typically within 1 kilobase) upstream of a cDNA encoding the gene. The availability of the complete sequence of the human genome permits the genomic region 5′ to any gene to be inspected for consensus promoter sequences, such as AT-rich sequences termed “TATA” boxes, and additional sequences comprising the sequence “CAAT” that are recognized as mediating the interaction of the nucleic acid of the promoter with protein factors such as RNA polymerase. Putative promoters can be readily tested by inserting the putative promoter sequence upstream from a reporter gene and comparing reporter gene activity in such constructs with activity in constructs without the putative promoter insert.

Recombinant expression constructs can be introduced into appropriate mammalian cells as understood by those with skill in the art, most preferably transfection and electroporation. Preferred embodiments of said constructs are produced in plasmid vectors or other vectors that can be used to easily produce useful quantities of the vector. Alternative embodiments include transmissible vectors, more preferably viral vectors and most preferably retrovirus vectors, adenovirus vectors, adeno-associated virus vectors, and vaccinia virus vectors, as known in the art. See, generally, MAMMALIAN CELL BIOTECHNOLOGY: A PRACTICAL APPROACH, (Butler, ed.), Oxford University Press: New York, 1991, pp. 57-84. Cells transiently transfected with the recombinant expression construct and more preferably cells stably transfected with the construct and selected using a selective agent are advantageously used in the practice of certain embodiments of the methods of the invention.

Detection of the senescence response in clinical cancers requires diagnostic markers for senescent cells. The most common senescence marker, SA-β-gal (Dimri et al., 1995, Proc. Natl. Acad. Sci. USA 92: 9363-9367), has two major disadvantages: it represents an enzymatic activity which is preserved only in frozen tissue samples and for a limited period of time, and it is not mechanistically related to growth arrest of senescent cells. The invention provides a number of genes that are upregulated in senescent cells. These proteins provide sensitive diagnostic markers for cytotoxic agent-induced senescence. Of special interest as diagnostic markers are several genes that are upregulated in senescent cells and are functionally related to growth arrest, such as EPLIN, BTG 1, BTG2, WIP 1, Maspin, MIC-1, IGFBP-6 and amphiregulin. Induction of these senescence-associated growth inhibitors is not limited to doxorubicin-treated HCT116 cells; for example EPLIN, a growth-inhibitory protein that was downregulated in 20 of 21 carcinoma cell lines relative to normal epithelial tissues (Maul et al., 1999, Oncogene 18: 7838-7841), is strongly induced in MCF-7 breast carcinoma cells by treatment with retinoids, under the conditions that produce senescence-like growth arrest (Dokmanovic et al., 2002, Cancer Biol. Ther. 1: 16-19 and in co-owned and co-pending U.S. Ser. No. 09/865,879, filed May 25, 2001, incorporated by reference herein). Retinoid treatment was also shown to induce a secreted growth inhibitor IGFBP-6 (Dailly et al., 2001, Biochim. Biophys. Acta 1518: 145-151). Most of the other senescence-associated growth inhibitors have been shown to be induced by DNA damage in a variety of other tumor-derived cell lines, including BTG1 (Cortes et al., 2000, Mol. Carcinogen. 27: 57-64), BTG2 (Fletcher et al., 1991, J. Biol. Chem. 266: 14511-14518), WIP1 (Fiscella et al., 1997, Proc. Natl. Acad. Sci. USA 94: 6048-6053), Maspin (Zou et al., 2000, J. Biol. Chem. 275: 6051-6054 and MIC-1 (Komarova et al., ibid.). However, none of these studies appreciated the association of these genes with senescence, and the general inducibility of such genes by DNA damage disclosed herein strongly indicates that such genes are broadly applicable markers of damage-induced senescence.

The invention also provides genes the expression of which is downregulated in cytotoxic agent-induced senescence. These genes are useful for detecting senescence in tumor cells in like manner as genes that are induced during senescence, except that senescence will be marked by downregulation of such genes. Several of these genes are of special interest as markers that are downregulated in senescent cells, including HFH-11 (Trident), a transcription factor implicated in cell cycle progression (Ye et al., 1999, Mol. Cell. Biol. 19: 8570-8580), STEAP, a gene overexpressed in different cancers (Hubert et al., 1999, Proc. Natl. Acad. Sci. USA 96: 14523-14528), RHAMM, shown to have oncogenic activity (Hall et al., 1995, Cell 82: 19-26) INSIG1, implicated in liver regeneration (Peng et al., 1997, Genomics 43: 278-284) and LRPR1 that mediates proliferative response to FSH (Slegtenhorst-Eegdeman et al., 1995, Mol. Cell. Endocrinol. 108: 115-24).

Changes in gene expression, either induction or repression and either native genes of reporter gene constructs as disclosed herein, are detected using methods well-established in the art. These include hybridization assays for detecting cellular nucleic acid, most preferably mRNA, said assays including northern hybridization, Southern hybridization, and any of a variety of in vitro amplification methods known in the art. Gene expression changes can also be detected using immunological reagents and methods, including enzyme-linked immunosorbent assay (ELISA) and other assays using polyclonal or monoclonal antibodies, antibody fragments or recombinant or chimeric antibodies and such immunological reagents. Activity of specific gene products, most preferably used with reporter gene constructs having known and quantifiable activities and most preferably producing easily-detected products are also advantageous for detecting senescence-associated changes in gene expression.

Elucidation of molecular changes associated with treatment-induced senescence is also advantageous therapeutically. Permanently arresting tumor cell growth through the induction of accelerated senescence is an attractive treatment approach, since this response to drug treatment can be elicited even under the conditions of minimal cytotoxicity. The instant disclosure that drug-induced senescence is associated with concerted induction of multiple antiproliferative genes (some of which also inhibit the growth of neighboring cells) suggests the existence of common regulatory pathways activating such genes. Importantly, most of the growth-inhibitory genes are also induced by doxorubicin treatment in p53-deficient cells. Agents that can be developed to stimulate the induction of senescence-associated growth-inhibitory genes are likely therefore to be efficacious against tumors with or without functional p53.

The obverse side of drug-induced senescence, however, is the induction of genes associated with pathological conditions (such as Alzheimer's disease), as well as proteases and mitogenic, antiapoptotic and angiogenic secreted factors. Expression of such genes by senescent cells may have potentially adverse effects in the short term (growth stimulation of non-senescent tumor cells) and in the long term (increased likelihood of de novo carcinogenesis and age-related diseases). A linkage between cell senescence and carcinogenesis in vivo has been suggested by a recent study of Paradis et al. (2001, Human Pathol. 32: 327-332), who found that SA-β-gal expression in normal human liver was strongly correlated with the development of hepatocellular carcinoma. Such linkage was also directly demonstrated by Krtolica et al. (2001, Proc. Natl. Acad. Sci. USA 98: 12072-12077), who found that mixing transformed epithelial cells with senescent (but not with pre-senescent) fibroblasts enhances the growth and tumorigenicity of the transformed cells. p21 induction upregulates many disease-associated genes and induces paracrine anti-apoptotic and mitogenic activities (Chang et al., 2000, ibid.), and p21 knockout was shown herein to decrease or delay the induction of such genes as prosaposin, TGFα and Alzheimer's βAPP. These observations suggest that p21-stimulated regulatory pathways may be largely responsible for the expression of disease-associated genes in senescent cells.

The present invention provides methods for identifying compounds that induce senescence in tumor cells without concomitantly inducing expression of said mitogenic, antiapoptotic and angiogenic secreted factors or genes associated with pathological conditions. The existence of such compounds is suggested by the behavior of retinoids, which induce tumor cell senescence through co-activation of several growth-inhibitory genes but not of p21 or other genes associated with pathological conditions (as disclosed in co-owned and co-pending U.S. Ser. No. 09/865,879, filed May 25, 2001, incorporated by reference herein and in Dokmanovic et al., 2002, Cancer Biol. Ther. 1: 16-19), and the present invention provides methods to identify other compounds dissociated from cytotoxicity or other confounding features of compounds known in the art to produce senescence in tumor cells.

The invention also provides methods for monitoring the efficacy of treatment, by identifying tumor cells that have become senescent and are no longer able to grow and distinguishing said cells from tumor cells that recover and proliferate after treatment. The detection of the markers of senescence in the biopsies of treated tumors can be used as an indicator of treatment response. This type of diagnostics should be useful in many clinical situations, including for example as a biopsy test to evaluate the success of radiation therapy that may potentially require several months or even years for complete response (see Cox et al., 1983, Int. J. Radiat. Oncol. Biol. Phys. 2: 299-303; Bataini et al., 1988, Am. J. Surg. 155: 754-760). The predominance of tumor cells that express markers of senescence is expected to be positively correlated with the success of treatment. Expression of the corresponding genes can be measured at the protein level, using antibodies against the corresponding gene products for in situ immunostaining, enzyme-linked immunosorbent assay (ELISA), or western blotting. Gene expression can also be measured at the nucleic acid level, most preferably by detecting expression of RNA encoding at least one of said genes, using in situ hybridization, in situ RT-PCR, or bulk RNA analysis techniques, such as RT-PCR or different forms of filter hybridization (including northern blotting). The choice of markers that are inhibited in senescent cells is provided by the genes listed in Table 1. The choice of senescence markers that are induced in senescent cells is provided by the genes listed in Table 2. Markers inhibited in senescent cells include the genes that are causally involved in cell proliferation and are known to be inhibited in other systems of cell senescence, including for example Ki-67 (which is already widely used as a proliferation marker), CENP-F, AIM-1, MAD-2, ribonucleotide reductase M1, and thymidine kinase. Such markers also include genes that show tumor-specific expression and have not been previously shown to be inhibited in senescence, such as STEAP, RHAMM or TLS/FUS. Of special interest as senescence markers are the genes that are induced in senescent cells and are causally involved in cell growth inhibition, including for example BTG1, BTG2, EPLIN, WIP1, Maspin, MIC-1, IGFBP-6 or amphiregulin, and other genes expression of which is downregulated in tumors relative to normal tissues, such as P-cadherin, desmoplakin, desmoyokin, and neurosin.

As disclosed herein, cytotoxic agent-inducible and repressible genes are useful targets for identifying compounds other than cytotoxic agents that mimic the physiologically-based growth inhibitory effect on cell proliferation. Identifying such compounds advantageously provides alternative agents for producing growth arrest in mammalian cells, particularly tumor cells and other cells that proliferate inappropriately or pathogenically. Such compounds are beneficial because they can mimic the growth-inhibitory effects of cytotoxic agents.

Another advantage of such compounds is that they can be expected to have a growth-inhibitory effect without producing systemic side effects found with other growth-inhibitory compounds known in the prior art. For example, many growth-inhibitory drugs and compounds known in the prior art disadvantageously induce p21 gene expression, which induces senescence, growth arrest and apoptosis by activating a plurality of genes, the expression of which is associated with the development of diseases, particularly age-related diseases such as Alzheimer's disease, atherosclerosis, renal disease, and arthritis (as disclosed in co-owned and co-pending U.S. Ser. No. 60/265,840, filed Feb. 1, 2001 (Attorney Docket No. 99,216-E) and U.S. Ser. No. 09/861,925, filed May 21, 2001 (Attorney Docket No. 99,216-F), incorporated by reference herein). Discovery of compounds that mimic the growth-inhibitory effects of cytotoxic agents chemotherapeutic drugs without producing the toxic side effects of growth-inhibitory compounds known in the art is advantageously provided by the invention.

Identification herein of cytotoxic agent-induced senescence-associated genes with pathogenic activity provides targets for developing drugs that inhibit the induction of such genes. The invention provides methods for assaying test compounds that inhibit induction of senescence-associated genes consequent to cytotoxic agent-induced senescence, by contacting cells with the test compound. Compounds that inhibit induction of these genes show no increased expression of these genes in agent-treated cells compared with untreated cells. Reporter gene constructs are also advantageously used to assay gene induction and lack thereof in the methods of the invention directed to these disease-associated genes.

The following Examples are intended to further illustrate certain preferred embodiments of the invention and are not limiting in nature.

### EXAMPLE 1

Permanent Growth Arrest in Tumor Cells Treated with a Cytotoxic Agent is Associated with the Development of a Senescent Phenotype

Cytological and gene expression analyses were performed to determine the effects of doxorubicin, a widely used anticancer drug that produces DNA damage by stabilizing a cleavable intermediate complex formed by topoisomerase II in the process of DNA segregation, on human colon cancer cells (HCT 116) in culture.

HCT116 colon carcinoma cells (Myohanen et al., 1998, Cancer Res. 58: 591-593; Accession No. CCL-247, American Type Culture Collection, Manassas, Va.), including wild-type, p21−/− (clone 80S4) and p53−/− (clone 379.2) cell lines (a gift of Dr. B. Vogelstein, Johns Hopkins University) were grown in Dulbecco Modified Eagle Medium with 10% FC2 serum. Cells were plated at 5×10⁶ cells per 15-cm plate and treated for 24-hr with 200 nM doxorubicin. Thereafter, cells were allowed to recover in drug-free media up to 10 days. For fluorescence-activated cell sorter (FACS) analysis of cell division, cells were labeled with PKH2 (a lipophilic fluorophore; Sigma Chemical Co., St. Louis, Mo.), which stably incorporates into the plasma membrane and distributes evenly between daughter cells, resulting in gradual decrease in PKH2 fluorescence during consequent cell divisions (Horan et al., 1989, Nature 340: 167-168). FACS analysis and cell sorting carried out as described in Chang et al. (1999, Cancer Res. 59: 3761-3767 and 1999, Oncogene 18: 4808-4818). Sorted fractions of senescent (PKH2^{hi}) and proliferating (PKH2^{lo}) cells (90-95% purity) were analyzed for DNA content using propidium iodide (PI) staining and FACS analysis as described by Jordan et al. (1996, Cancer Res. 56: 816-825). The cells were also stained for senescence-associated β-galactosidase (SA-β-gal) activity as described by Dimri et al. (1995, Proc. Natl. Acad. Sci. USA 92: 9363-9367). Finally, clonogenicity of the sorted populations was tested by plating 2,000-10,000 sorted cells per 10-cm plate.

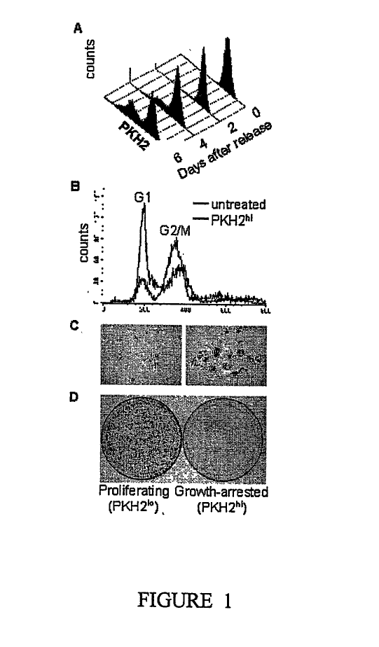

The results of these assays are shown in FIGS. 1A through 1D. Cell proliferation as detected by FACS using PKH2 fluorescence is shown in FIG. 1A. Changes in PKH2 fluorescence were monitored by FACS on different days after doxorubicin treatment. Cells that died after drug treatment were excluded from this analysis based on their staining with membrane-impermeable dye PI. Almost all PI-negative cells remained growth-arrested (PKH2^{hi}) for the first 2-3 days after doxorubicin treatment, but a proliferating cell population (PKH2^{lo}) emerged starting from day 4. A substantial fraction of cells, however, remained PKH₂^{hi} and did not decrease their fluorescence, indicating that these cells did not divide even once after release from the drug. 6-10 days after doxorubicin treatment, the surviving cells were separated by FACS into PKH₂^{hi} and PKH2^{lo} fractions.

DNA content analysis showed that most of PKH2^{hi} cells remained in G2, the phase where most of the cells had been originally arrested by doxorubicin through its effect on topoisomerase II (FIG. 1B). As shown in FIG. 1C, PKH2^{hi} cells were greatly enlarged and stained positively for SA-β-gal, indicating their senescent phenotype. In contrast, PKH2^{lo} cells retained normal size and remained negative for SA-β-gal. The ability to form colonies was essentially confined to the PKH2^{lo} fraction (FIG. 1D), indicating that the senescent PKH2^{hi} cells have lost their proliferative capacity.

These results clearly indicated that HCT 116 cells were separated into two different populations following doxorubicin treatment: a senescent cell population and a population that regained the capacity to proliferate.

### EXAMPLE 2

Identification of Genes Induced and Repressed in Doxorubicin-Induced Senescence

The populations of senescent and proliferating cells produced by doxorubicin treatment of HCT 116 cells as described in Example 1 were used to identify differences in gene expression between these cell populations and untreated cells.

In these experiments, poly(A)⁺ RNA and protein extracts were prepared from PKH2^{lo} and PKH2^{lo} cell populations, separated in different experiments 6, 9 or 10 days after release from doxorubicin. Fluorescent cDNA probes were synthesized and used for hybridization with the Human UniGEM V 2.0 cDNA microarray and signal analysis (assays were conducted by IncyteGenomics, St. Louis, Mo., as described at that company's web site, www.incyte.com). Changes in gene expression were verified by semi-quantitative reverse transcription-PCR (RT-PCR), essentially as described (Noonan et al., 1990, Proc. Natl. Acad. Sci. USA 87: 7160-7164), using β-actin as an internal normalization standard and the oligonucleotide primers shown in Table 3. RT-PCR analysis was carried out using two pairs of proliferating- and senescent-cell RNA preparations isolated in independent experiments, with the same results; for a subset of the genes, the assays were reproduced with the same pair of RNA samples. These results were confirmed by immunoblotting assays that were carried out at least twice (with the same results), using the following primary antibodies: mouse monoclonal antibodies against β-actin (Sigma Chemical Co.), p53 and p21 (Oncogene Research, Cambridge, Mass.), Maspin (Pharmingen, San Diego, Calif.), keratin 18 (Neomarkers, Union City, Calif.), cyclin D1 (Santa Cruz Biotechnology, Santa Cruz, Calif.), and rabbit polyclonal antibodies against ATF-3 (Santa Cruz), Mad-2 (BabCo, Richmond, Calif.) and EPLIN (a gift of Dr. D. Chang, UCLA). Bands were detected using horseradish peroxidase-labeled secondary antibodies and ECL chemiluminescence detection kit (Amersham Pharmacia Biotech, Piscataway, N.J.).

Fluorescent cDNA probes were prepared from RNA of senescent (PKH2^{hi}) and proliferating (PKH2^{lo}) cell populations and used for differential hybridization with UniGEM V 2.0 human cDNA microarray (IncyteGenomics, Inc.), containing >9,000 genes. 82% of the more than 9,000 sequence-verified genes and expressed sequence tags (ESTs) present in the UniGem V 2.0 microarray gave measurable hybridization signals with both probes. Lists of genes identified by this hybridization as downregulated or upregulated in the senescent relative to proliferating cells (with balanced differential expression of 2.0 or higher) are provided in Tables 1 and 2.

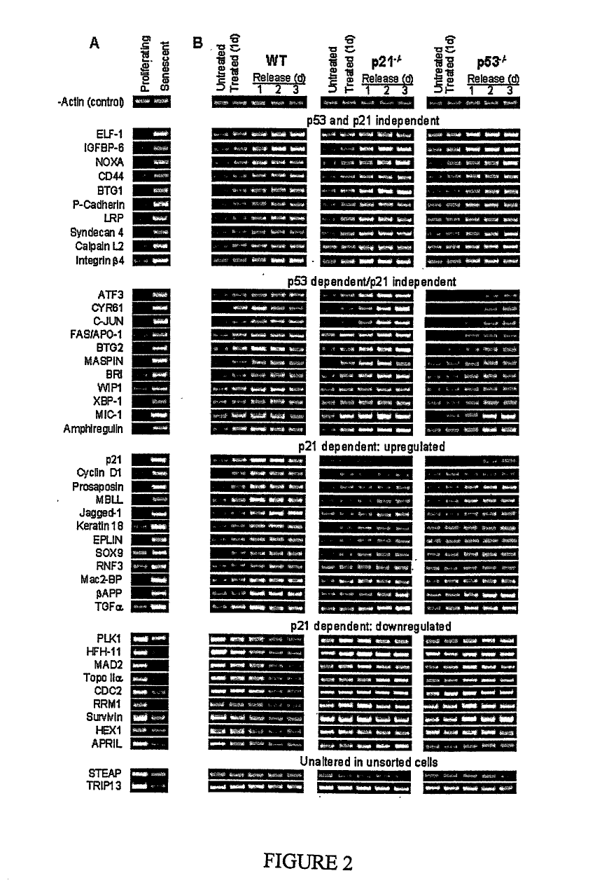

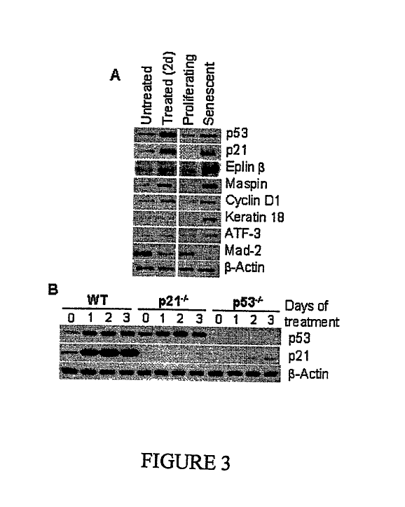

RT-PCR analysis (FIG. 2A) was carried out for 74 individual genes detected using the hybridization assay and confirmed qualitative changes in gene expression for 26/29 downregulated and 37/45 upregulated genes. In most cases, differences in gene expression revealed by RT-PCR were much higher than the values indicated by cDNA microarray hybridization. Changes in the expression of 7 genes were also confirmed at the protein level by immunoblotting (FIG. 3A).

More than one half of 68 genes and ESTs downregulated in senescent cells are known to play a role in cell cycle progression: 25 of these genes are involved in different stages of mitosis or DNA segregation (e.g., CDC2, Ki-67, MAD2, Topoisomerase IIα); 11 genes function in DNA replication and chromatin assembly (e.g. ribonucleotide reductase M1, thymidylate kinase, replication protein A3); and 4 genes are involved in DNA repair (e.g. HEX1, FEN1). Downregulation of genes involved in cell proliferation correlates with the growth-arrested state of senescent cells and demonstrates the biological relevance of gene expression profiling in our system.

In addition, multiple growth-inhibitory genes were induced by doxorubicin treatment. Senescent HCT116 cells were found to upregulate multiple genes with documented growth-inhibitory activity, providing an ample explanation for the maintenance of doxorubicin-induced cell cycle arrest in the absence of p16 (which is not expressed in HCT 116 cells). One of the upregulated genes is p21 (shown in FIG. 2A). Analysis of p21 and p53 protein induction by doxorubicin in wild type, p53−/− (14) and p21−/− (15) HCT116 cell lines demonstrated that p21 induction in this system is strongly dependent on p53 (shown in FIG. 3B). Both p53 and p21 proteins are maintained at elevated levels in senescent cells isolated 9 days after release from the drug (FIG. 3A). In contrast to p21, however, p53 is upregulated only at the protein level.

In addition to sustained p21 induction, senescent cells strongly overexpress many other growth inhibitors, including several known or putative tumor suppressor genes. Some of these genes encode intracellular growth-inhibitory proteins, including tumor suppressor BTG1 and its homolog BTG2, putative tumor suppressor EPLIN (Epithelial Protein Lost in Neoplasm) and WIP1 phosphatase. Senescent HCT116 cells also overexpress several secreted growth inhibitors, including MIC-1 (pTGF-β), insulin-like growth factor binding protein-6 (IGFBP-6), serine protease inhibitor Maspin (a tumor suppressor downregulated in advanced breast cancers), and amphiregulin, an EGF-related factor that inhibits proliferation of several carcinoma cell lines while promoting the growth of normal epithelial cells. These findings suggest that drug-induced growth arrest of tumor cells is maintained by a set of apparently redundant intracellular and paracrine factors.

Differences in gene expression between senescent and proliferating populations of drug-treated HCT116 cells parallel the differences between normal and cancerous epithelial cells. In addition to the above listed tumor suppressors, senescent HCT116 cells induce several other genes that are downregulated in cancers relative to normal epithelial cells (including MIC-1, P-cadherin, desmoplakin, desmoyokin, neurosin). On the other hand, senescent cells downregulate not only multiple genes involved in cell proliferation but also some other genes that have oncogenic activity (RHAMM and TLS/FUS) or show tumor-specific expression (STEAP). Another sign of putative “normalization” of senescent cells is the upregulation of six members of the keratin gene family. The strongest induction in this group was observed for keratins 8 and 18, a keratin pair with anti-apoptotic activity (Caulin et al., 2000, J. Cell Biol. 149: 17-22). However, senescent HCT116 cells show no evidence of apoptosis, even though they upregulate two proapoptotic genes, APO-1/Fas and NOXA.

In addition to the growth-inhibitory genes, senescent HCT116 cells show increased expression of genes for secreted mitogenic, anti-apoptotic and angiogenic factors, such as extracellular matrix (ECM) proteins Cyr61 and prosaposin, and transforming growth factor α (TGF-α). Induction of such genes results in paracrine activities, which promote tumor cell growth in vitro and in vivo. Such activities have been previously associated with replicative senescence (Campisi, 2000, In vitro 14: 183-188) in normal cells, and with p21 induction in tumor cells (Chang et al., 2000, Proc. Natl. Acad. Sci. USA 97: 1497-150117). Senescent HCT 116 cells also upregulate several proteases (kallikrein-7, calpain L2, neurosin, urokinase-type plasminogen activator) that may potentially contribute to metastatic growth. Several other genes induced in senescent HCT 116 cells are involved in cell adhesion and cell-cell contact (including P-cadherin, Mac2-binding protein and desmoplakin). Other induced genes encode ECM receptors, including several integrins and syndecan-4 (ryudocan), involved in angiogenesis. Some other transmembrane proteins induced in senescent cells are growth-regulatory proteins CD44 and Jagged-1, Alzheimer's β-amyloid precursor protein (βAPP), and another amyloid precursor, BRI, associated with an Alzheimer-like disease. Altogether, secreted factors, ECM proteins, ECM receptors and other integral membrane proteins make up 33 of 68 genes with known functions that are induced in senescent HCT116 cells. In contrast, only 2 of 64 downregulated genes with known function were induced in the senescent cell population of HCT 116 cells treated with doxorubicin.

One class of genes that are differentially expressed in doxorubicin-treated HCT 116 cells are genes encoding known or putative transcription factors or cofactors. Genes for several known or putative transcription factors and cofactors show altered regulation in the senescent HCT116 cells. One of the downregulated transcription factors is winged helix protein HFH-11 (Trident), a positive regulator of DNA replication, that is specifically expressed in cycling cells (Ye et al., 1999, Mol. Cell. Biol. 19: 8570-8580). Several upregulated transcription factors are related to the AP-1 family, which mediates cellular responses to various mitogenic signals, interferons and different forms of stress (Wisdom, 1999, Exp. Cell. Res. 253: 180-185). These include c-Jun and two other basic leucine zipper proteins, XBP-1 (structurally related to c-Jun) and ATF3 that dimerizes with c-Jun. Sustained upregulation of ATF3 mRNA and protein in senescent cells is surprising, since induction of this stress-responsive factor is usually transient (over hours), due to the ability of ATF3 to inhibit its own transcription (Wolfgang et al., 2000, J. Biol Chem. 275: 16865-16870). Another induced transcription factor is ELF-1, a member of Ets family of helix-loop-helix proteins that are known to interact functionally, and possibly physically, with AP-1 (Wisdom, ibid.).

The observed pattern of gene expression in cytotoxic drug-induced senescence of HCT116 cells showed many similarities to senescence in normal cells. Some of the general properties of senescent cells (other than terminal growth arrest) are resistance to apoptosis, increased cell adhesion (associated with overproduction of ECM components), and secretion of proteases, protease inhibitors, and mitogenic factors (Campisi, ibid.). Genes involved in all of these phenomena are amply represented among those that are upregulated in senescent HCT 116 tumor cells. In contrast to normal cells, however, senescent HCT116 cells don't upregulate p16 or tumor suppressor PML associated with RAS-induced accelerated senescence (Pearson et al., 2000, Nature 406: 207-210).

Changes in gene expression associated with drug-induced senescence also show parallels with organism aging. Some of the proteins that are induced in the senescent HCT116 colon carcinoma cells, such as βAPP and prosaposin, show age-dependent expression in animals. Remarkably, Maspin, CD44 and Cyclin D1 were reported to be upregulated specifically in the colonic epithelium of aging animals (Lee et al., 2001, Mech Ageing Dev. 122: 355-371). In addition, eight genes downregulated in senescent HCT116 cells also showed decreased expression in actively growing fibroblast cultures from old people relative to similar cultures from young people, whereas two induced genes (MIC-1 and desmoplakin) were upregulated in cultures from older individuals (Ly et al., 2000, Science 287: 2486-2492). These results demonstrate that the process of drug-induced senescence in tumor cells is related to both replicative senescence and organism aging.

### EXAMPLE 3

Effects of p53 and p21 Knockout on Cytotoxic Drug-Induced Changes in Senescence-Associated Gene Expression

Many of the genes that show altered expression in senescent HCT116 cells have shown similar changes upon overexpression of p53 (9 downregulated and 11 upregulated genes) or p21 (46 downregulated and 7 upregulated genes) (see Tables 1 and 2). p53 acts as a direct transcriptional activator of many genes (including p21) and indirectly regulates a group of genes that do not have p53-binding sites in their promoters (Komarova et al., 1998, Oncogene 17: 1089-1096; Zhao et al., 1999, Cell Res. 9: 51-59). A prominent class of p53-induced genes encode secreted growth-inhibitory factors, providing paracrine antiproliferative activity (Komarova et al., ibid.). In contrast to p53, p21 is not a transcriptional regulator per se, but it interacts with a broad network of transcription factors, cofactors and mediators of signal transduction (Dotto, 2000, Biochim. Biophys. Acta 1471: M43-M56). Overexpression of p21 in fibrosarcoma cells results in downregulation of multiple cell proliferation genes and upregulation of many ECM components and secreted mitogenic and antiapoptotic factors, providing the corresponding activities in conditioned media of p21-induced cells (Chang et al., 2000, ibid.). A known mechanism for transcription activation by p21 is based on its ability to stimulate p300/CBP transcription cofactors (Snowden et al., 2000, Mol. Cell. Biol. 20: 2676-2686). HCT116 cells, however, express a dominant mutant form of transcription factor p300 (Gayther et al., 2000, Nat. Genet. 24: 300-303), which may explain why senescent HCT116 cells upregulate a relatively small number of p21-inducible genes.

To elucidate the roles of p53 and p21 in the observed changes in gene expression, expression of senescence-associated genes upon doxorubicin treatment of wild type, p21−/− and p53−/− HCT116 cells was analyzed. RNA samples were isolated before the addition of the drug, immediately after one-day treatment with doxorubicin, and on three consecutive days after the removal of the drug. Expression of 33 genes that were upregulated and 11 genes downregulated in senescent cells was analyzed by RT-PCR as described above; results are shown in FIG. 2B.

This analysis showed that all the tested genes were expressed in the untreated wild-type cells at levels similar to those in the proliferating fraction of doxorubicin-treated cells. Senescence-associated changes in the expression of most of these genes became detectable in the total population of wild-type HCT116 cells after one-day doxorubicin treatment or one day after release from the drug. This early response made it possible to evaluate the effects of p21 and p53 knockouts on total populations of doxorubicin-treated cells, without having to purify the small senescent fractions of p21−/− and p53−/− cell lines.

Approximately one third of the genes that are upregulated in senescent cells showed almost indistinguishable response among the wild-type, p21−/− and p53−/− cell lines, indicating that the induction of these genes does not involve either p53 or p21 (FIG. 2B). These genes include tumor suppressor BTG1 and secreted growth inhibitor IGFBP-6. Surprisingly, one of the genes that shows no p53 dependence is NOXA, although it is known to be inducible by p53. The remaining two thirds of the upregulated genes showed diminished or delayed induction in p53−/− cells. About one half of the latter genes were unaffected by p21 knockout. This group includes transcription factors of the AP-1 family, CYR61, and several intracellular (BTG2, WIP1) and secreted growth inhibitors (Maspin, MIC-1, amphiregulin). None of these genes, however, are completely dependent on p53 for their induction, and all of them are induced in p53−/− cells two days after release from the drug. Almost all senescence-associated growth inhibitors (except for p21 and EPLIN) are eventually induced in p53−/− cells to a level comparable to the wild-type cell line (FIG. 2B). These results provide an explanation for the diminished but still substantial induction of senescence-like growth arrest in p53−/− cells after doxorubicin treatment (Chang et al., 1999a, ibid.).

A final group of the induced genes shows much weaker changes in p21−/− than in the wild-type cells (FIG. 2B), indicating that regulation of these genes is mediated through p21. Since p21 induction in doxorubicin-treated HCT116 cells is p53-dependent, such genes also show diminished induction in p53−/− cells. The strongest p21 dependence among the tested genes is found for Cyclin D1. None of p21-dependent genes produces secreted growth inhibitors, but two of them encode secreted mitogenic/antiapoptotic proteins (prosaposin and TGFA). Most of the genes that are downregulated in senescent HCT116 cells are known to be inhibited by p21 (Caulin et al., ibid.). In agreement with this observation, such genes show decreased expression after doxorubicin treatment only in the wild-type but not in p21−/− or p53−/− cell lines (FIG. 2B). Together with the genes that show p21-dependent induction, 20 of 31 tested genes that are affected by p53 knockout (excluding p21) are also affected to the same or greater degree by the knockout of p21. Therefore, p21, which until recently was not known to play a role in the regulation of gene expression, appears to be a major mediator of the corresponding effects of p53.

These results indicate that the genes identified herein can be used as markers for assessing compounds for their effects on cellular senescence and also for identifying compounds that induce the senescence phenotype by mechanisms that do not implicate p53, p21 or both.

### EXAMPLE 4

Construction of Promoter-Reporter Gene Constructs that are Induced in Senescent Cells and Screening for Agents that Induce Senescence in Tumor Cells

A cell-based screening assay is used to identify compounds that activate senescence-associated growth-inhibitory genes in p53-deficient tumor cells, without concurrent activation of secreted tumor-promoting factors. For this purpose, promoter constructs of different senescence-associated growth-inhibitory genes are constructed that drive expression of a chimeric GFP-luciferase reporter. Such a chimeric reporter was shown to be suitable for selection based on GFP fluorescence and for sensitive promoter activity measurements based on luciferase chemiluminescence (Kotarsky et al., 2001, Anal. Biochem. 288: 209-215). A similar chimeric reporter gene is commercially-available (Clontech, Palo Alto, Calif.). The promoter-reporter constructs are tested for inducibility by doxorubicin under conditions that activate the corresponding genes. The best-regulated promoter constructs are used to develop stably transfected cell lines, and cell lines identified that have the strongest induction of the reporter gene under conditions of drug-induced senescence.

Once suitable reporter cell lines are developed, optimized conditions for high throughput screening (HTS) of chemical libraries are determined based on luciferase activity of the reporter. This HTS assay is used to screen a chemical compound library (such as the Diversity Set of 1,990 compounds from the Developmental Therapeutics Program (DTP) of NCI). Positive compounds in this assay are then tested for their effects on expression of other genes associated with positive and negative aspects of accelerated senescence.

Seven senescence-associated growth-inhibitory genes are preferential targets for induction assays:

BTG1 is a tumor suppressor rearranged in t(8;12)(q24;q22) chromosomal translocation of B-cell leukemia and an inhibitor of cell proliferation (Rouault et al., 1992, EMBO J. 11: 1663-1670). BTG1 was shown to be induced by DNA damage in different human tumor cell lines (Cortes et al., 2000, Mol. Carcinog. 27: 57-64). Damage-induced BTG1 expression was shown by Cortes et al. and is shown herein to be independent of p53.

BTG2 (PC3/TIS21) is a BTG1 related antiproliferative gene (Rouault et al., 1996, Nat. Genet. 14: 482-486). BTG1 is stress-responsive (Fiedler et al., 1998, Biochem. Biophys. Res. Commun. 249: 562-565) and is also induced in different cell lines by DNA damage, growth factors and tumor promoters (Fletcher et al., 1991, J. Biol. Chem. 266: 14511-14518). BTG2 was shown to be induced by p53 at the level of transcription (Rouault et al., 1996, ibid.), but it is inducible by doxorubicin in p53−/− cells, albeit to a lesser degree than in the wild type cells.

IGF-binding protein 6 (IGFBP-6), a secreted inhibitor of IGF function and tumor cell growth (Bach, 1999, Horm. Metab. Res. 31: 226-234; Sueoka et al., 2000, Oncogene 19: 4432-4436), was shown to be inducible by retinoids at the level of transcription (Dailly et al., 2001, Biochim. Biophys. Acta 1518: 145-151). IGFBP-6 induction by doxorubicin shows no dependence on p53.

Amphiregulin is an EGF-related factor that was shown to inhibit the growth of several carcinoma cell lines, while promoting the growth of normal epithelial cells (Plowman et al., 1990, Mol. Cell Biol. 10: 1969-1981). Amphiregulin is the major target of transcriptional induction by WT1 Wilms tumor suppressor gene (Lee et al., 1999, Cell 98: 663-673) and is inducible by vitamin D3 (Akutsu et al., 2001, Biochem. Biophys. Res. Commun. 281: 1051-1056). Amphiregulin induction by doxorubicin shows only moderate dependence on p53.

MIC-1 (pTGF-β/PLAB/PDF/GDF15), a secreted growth-inhibitory member of TGF-β superfamily, was shown to be induced by p53 at the level of transcription (Tan et al., 2000, Proc. Natl. Acad. Sci. USA 97: 109-114) and was suggested to be a key mediator of paracrine growth-inhibiting effects of p53 (Kannan et al., 2000, FEBS Lett. 470: 77-82). Surprisingly, MIC-1 induction by doxorubicin shows only weak dependence on p53.

Maspin, a secreted serine protease inhibitor, has been identified as a tumor suppressor whose expression is lost in many advanced breast cancers (Domann et al., 2000, Int. J. Cancer 85: 805-810). Maspin shows very strong induction by DNA damage at the protein level, in doxorubicin-treated HCT116 cells, and others; Zou et al. (2000, J. Biol. Chem. 275: 6051-6054) showed maspin induction by drug treatment in four other tumor cell lines. Maspin expression is induced at the transcriptional level by p53 (Zou et al., ibid.). Although p53 knockout strongly decreases Maspin induction by doxorubicin, such induction is still readily detectable in p53−/− cells.

EPLIN (Epithelial Protein Lost in Neoplasms), an actin-binding cytoskeletal protein, is expressed in almost all normal epithelial tissues but downregulated in 20 of 21 tested carcinoma cell lines. EPLIN inhibits cell proliferation, making it a putative tumor suppressor (Maul & Chang, 1999, Oncogene 18: 7838-7841). EPLIN is induced not only in the senescent population of doxorubicin-treated HCT116 cells, but also in MCF7 breast carcinoma cells that undergo senescence-like growth arrest upon treatment with retinoids. Among all the genes in this group, EPLIN shows the weakest induction by doxorubicin in the unsorted cells; this induction is even further diminished in p21−/− and p53−/− cells. Although this pattern makes it potentially difficult to detect EPLIN induction upon drug treatment, strong increase in EPLIN expression in the sorted population of senescent cells suggests that its induction may be a particularly specific marker of senescence.

Functional promoter sequences have been published for all of these genes: BTG1 (Rodier et al., 1999, Exp. Cell Res. 249: 337-348), BTG2 (Fletcher et al., 1991, ibid.), IGFBP-6 (Dailly et al., 2001, ibid.), amphiregulin (Lee et al., 1999, ibid.), Maspin (Zou et al., 2000, ibid.), MIC-1 (Tan et al., 2000, ibid.), EPLIN (Gao et al., 2000, J. Cell Physiol. 184: 373-379; EPLIN has two alternative promoters; the preferred promoter is the promoter corresponding to the longer β isoform that is preferentially expressed in HCT116 and MCF7 cells. Transcription of the corresponding genes is regulated by various factors (DNA damage, serum, differentiating agents, phorbol esters, tumor suppressors) through cis-regulatory elements in their promoters. In addition, Maspin has been shown to be silenced in breast cancers at the level of promoter methylation (Domann et al., 2000, ibid.). Thus, it can be expected that senescence-associated changes in the expression of these genes will be reproducible in promoter constructs. Substantially all of these promoters share several common cis-regulatory sites, including AP-1, AP-4, ELK1 and GATA as revealed by examination of transcription factor binding sites in the corresponding promoter sequences, using MatInspector V2.2 program based on TRANSFAC 4.0 database. Together with the observed coregulation of these genes in drug-induced senescence, these observations support the likelihood of identifying agents that will stimulate all or most of these genes at the same time.

Reporter gene constructs are prepared by traditional cloning methods or by polymerase chain reaction (PCR) amplification of promoter sequences using primers designed from sequences flanking the corresponding promoters and human genomic DNA as a template. The promoter sequences are cloned upstream of a suitable reporter gene, the most convenient of which is useful both as a selectable marker and as the basis for HTS. A commercially-available reporter comprising a chimera of green fluorescence protein and luciferase is most suitable for this purpose. This reporter is a chimeric protein formed by the Enhanced form of Green Fluorescent Protein (GFP) (commercially-available from Clontech) at the amino terminal end, fused with firefly luciferase at the carboxyl terminus. This chimeric reporter provides strong GFP fluorescence and high sensitivity of luciferase-based chemiluminescence assays. This gene is cloned into a promoterless vector in an orientation that a convenient cloning site or multiple cloning site is operably linked at the 5′ end of the reporter gene, so that the promoters from the seven senescence-associated genes can be easily inserted into and thereby operably linked to the reporter gene.

Several of the tested genes are known to be inducible by p53. To select compounds that activate senescence-associated growth inhibitors through p53-independent mechanisms, p53-deficient cell lines will be used for screening. The primary cell line is a p53−/− derivative of HCT116 colon carcinoma cells (Bunz et al., 1998, Science 282: 1497-1501), as described above. Promoter constructs that show positive results in this cell line are then tested in other p53-deficient tumor cell types, to confirm that the induction of these promoters is not unique to HCT116 cells (damage-responsiveness in a number of cell lines has already been demonstrated for BTG1, BTG2 and Maspin, and retinoid inducibility in breast carcinoma lines was shown for EPLIN and IGFBP-6). p53-mutated tumor cell lines are used, particularly those cell lines that develop the senescent phenotype upon doxorubicin treatment, including SW480 colon carcinoma, U251 glioma and Saos2 osteosarcoma (as disclosed by Chang et al., 1999b, ibid.). Also used in these assays is a derivative of HT1080 fibrosarcoma wherein p53 function has been fully inhibited with a p53-derived genetic suppressor element GSE56 (as disclosed by Chang et al., 1999a, ibid.).

These promoter-reporter constructs are used initially in transient transfection assays for the induction of luciferase activity by doxorubicin treatment. For normalization, the tested constructs are mixed with a construct carrying a different reporter gene under a constitutively expressed promoter (e.g. β-galactosidase transcribed from the CMV promoter). These mixtures are transfected (using electroporation) into p53−/− HCT116 cells, which are then either untreated or treated with 200 nM doxorubicin. The activity of firefly luciferase and the control reporter gene (β-gal) are determined using commercially available assay kits, and the normalized values of firefly luciferase activity are compared between the treated and untreated cells. The promoter constructs that provide the highest expression and the best induction are determined from these assays.

Several promoter constructs showing at least 3-fold induction in transient transfection assays are transfected into p53−/− HCT116 cells, and stably transfected cell lines selected with puromycin. About 100 clonal transfectants from each tested construct are isolated and expanded to the size of close to 100,000 cells. At this stage, the picked lines are screened for activity by doxorubicin in 96-well plate assays (as described in more detail below). The best-inducible cell lines are expanded and subsequently characterized by repeated testing for both GFP and luciferase induction. Reporter cell lines are selected to maximize the absolute level of induced luciferase expression while retaining a high fold-induction, because high absolute luciferase expression minimizes the number of cells required to produce a detectable signal in HTS assays. Once developed, these optimal cell lines are analyzed with regard to the time course and doxorubicin dose-dependence of reporter expression and tested to verify senescence-specificity of the expression. The latter analysis is performed by labeling cells with PKH26 (a fluorophore related to PKH2 but having a red-shifted emission wave length), followed by doxorubicin treatment and release into drug-free media. Between 6-7 days after release, cells are analyzed with FACS by two-color analysis for PKH26 and GFP fluorescence. GFP fluorescence selectively associated with PKH26^{hi} (senescent) cells is thereby determined without physical sorting. Finally, reporter expression inducibility in the selected cell lines is tested with senescence-inducing agents other than doxorubicin, other agents, such as ionizing radiation, cisplatin, aphidicolin or cytarabine (Chang et al., 1999b, ibid.). The primary reporter cell line for subsequent compound screening is generated thereby, and secondary cell lines expressing the reporter from the promoters of different genes can be used for confirmatory assays.

The primary reporter cell line developed as described above is used to develop HTS assay. The dual GFP-luciferase nature of the reporter gene is especially convenient for conducting screening assays using the more sensitive luciferase-based chemiluminescence assay, and the GFP fluorescence to confirm that the effect of a tested compound is not due to artifactual influence on the luciferase assay.

Primary screening will be carried out using the assay conditions established for doxorubicin and other senescence-inducing agents, and similarly to the procedures used by other investigators for luciferase-based screening of chemical libraries (Sohn et al., 2001, Ann. Surg. 233: 696-703). Aliquots of 1 mM stocks of each compound in a compound library are added at a final 2 μM concentration into a set of three 96-well plates containing cell culture media. These 96-well plates are then seeded with reporter cells and incubated at 37° C. for the required period of time, with at least two reagent-free negative control wells and two doxorubicin-containing positive control wells per plate. After incubation, the plates are read (with no further manipulations) in the fluorescence reader, to identify the wells with substantial increase in GFP activity. The same plates are then processed for luciferase assay and read in a microplate luminometer. Luminometer readings on three plates are used to identify candidate positives, and compared with the results of GFP fluorescence. Positive compounds are re-tested in another set of assays prior to secondary screening. The nature of the assays for increased luciferase and GFP activity, which need to be expressed in live cells over the course of the assay, should eliminate highly cytotoxic compounds from the list of candidates.

Compounds that score as positive in the primary analysis are tested for their effect on the expression of different senescence-associated genes. Some of these assays are carried out using stably transfected cell lines, where the reporter gene is driven by promoters of other genes than the one in the primary reporter line. These simple reporter activation assays are warranted if a very large number of positives are detected in the primary assay. A second reporter line can be used to limit the number of compounds to those that are active with more than one promoter. If the number of positive compounds after the primary screen is low, however, this secondary screening step is unnecessary and the positive compounds are used for direct analysis of the compounds on gene expression.

On addition and prior to extensive further screening the minimal concentration of the compound that produces a strong increase in the reporter assay is determined. This concentration is also tested on p53−/− HCT116 cells for its effect on the expression of the endogenous senescence-associated genes. In these assays, RNA is extracted before and after treatment, and expression of different senescence-associated genes is analyzed, for example by quantitative RT-PCR (as disclosed by Noonan et al., 1990) that allows expression levels for multiple genes among a set of RNA samples to be compared. A single RT-PCR assay uses about 50 ng of total cellular RNA, which makes it possible to carry out about 100 assays starting from 5 μg of total RNA, an amount that is typically used for a single lane in northern hybridization. In this assay, β-actin is used as a normalization standard, since its expression is unaltered in senescent cells, according to northern and western blots.

RT-PCR primers and assay conditions for 63 genes that are up- or downregulated in doxorubicin-induced accelerated senescence (Chang et al., 2001, ibid.) are disclosed in Table 3. These assays are used to test if the positive compounds can activate not only the growth-inhibitory genes that are described above, but also other senescence-associated growth regulators, such as WIP1, CD44, Jagged1, and also several genes that are known to be downregulated in cancers relative to normal cells and then upregulated in senescent tumor cells, such as P-cadherin, desmoplakin and desmoyokin. The latter genes are likely to be co-regulated with senescence-associated growth inhibitors that are downregulated in cancers (such as EPLIN or Maspin). On the other hand, it is expected that compounds will be found that will not induce p21 or the potentially pathogenic proteins that are upregulated in doxorubicin-induced senescence, such as secreted tumor-promoting factors TGFα, CYR61 and prosaposin, proteases such as kallikrein-7 or calpain L2, and plaque-forming proteins, such as Alzheimer's β-amyloid precursor and BRI. Positive compounds are also assayed for the effects of the compounds on genes that are downregulated in senescent cells, such as tumor-specific transmembrane protein STEAP, and genes involved in cell proliferation (e.g. Ki-67, Topoisomerase IIα, CDC2, PLK1, MAD2, Thymidylate synthetatse, Ribonucleotide reductase M1). Inhibition of the latter genes will be indicative of a cytostatic effect of the tested compound, which will be tested in separate assays (see below).

If this analysis reveals a compound that has the desired effect on gene expression, analyses are performed to determine how the compound affects cell growth. This analysis will be carried out both by standard cell proliferation assays, and by an assay that evaluates the cytostatic and cytotoxic components of the antiproliferative effect. In this assay, cells are labeled with PKH2, treated with the test compound either continuously or for a limited period of time (e.g. 24 hrs), and analyzed after the period of time corresponding to three cell doublings. For this analysis, attached and floating cells will be combined and stained with propidium iodide (PI), which stains only membrane-compromised (dead) cells. The stained cells are then analyzed by FACS for changes in PKH2 fluorescence and for the fraction of PI-positive cells, next to the control sample of untreated cells that were labeled with PKH2 at the same time. Increased PKH2 fluorescence relative to control cells indicates the inhibition of -cell division (cytostatic effect) and increased PI+ fraction indicates the cytotoxic effect. Compounds with preferentially cytostatic (rather than cytotoxic) effect on tumor cells are of particular interest, because such an effect is expected from the specific activation of the senescence program.

If a prototype compound with desired properties is found, a library of derivatives from this compound is prepared, which is then screened to find more effective agents. Such agents are evaluated as prototype drugs by preclinical studies.

### EXAMPLE 5

Construction of Promoter-Reporter Gene Constructs and Screening for Agents that Prevent the Induction of Pathogenic Genes Associated with Anticancer Agent-Induced Senescence

The results disclosed herein show that certain genes are induced by treatment with cytotoxic drugs that have been associated with diseases of aging and paracrine growth-stimulating effects, especially tumor cell growth stimulation. These genes include cyclin D1, serum-inducible kinase, CYR61, prosaposin, transforming growth factor α (TGFα), kallikrein 7, calpain-L2, neurosin, plasminogen activator, urokinase, amyloid beta (A4) precursor protein (βAPP), and integral membrane protein 2B (BRI/ITM2B). Promoters from these genes can be used to make reporter gene constructs in like manner as disclosed in Example 4 for other senescence-associated genes. These constructs can then be used to assay reporter gene induction by cytotoxic drug treatment in the presence and absence of a test compound.

Functional promoter sequences have been published for all of these genes: cyclin D1 (Motokura & Arnold, 1993, Genes Chromosomes Cancer 7: 89-95); CYR61 (Latinkic et al., 1991, Nucleic Acids Res. 19: 3261-7); prosaposin (Sun et al., 1998, Gene 218: 23-34); transforming growth factor a (TGFα; Raja et al., 1991, Mol. Endocrinol. 5: 514-20); kallikrein 7 (Yousef et al., 2000, Gene 254: 119-128); calpain-L2 (Suzuki et al., 1995, Biol Chem Hoppe Seyler. 376: 523-9); plasminogen activator urokinase (Riccio et al., 1985, Nucleic Acids Res. 13: 2759-71); and amyloid beta (A4) precursor protein (βAPP; Lahiri & Robakis, 1991, Brain Res. Molec. Brain Res. 9: 253-257).

Reporter gene constructs are prepared by modification of the methods described in Example 4. Senescence is induced in transient and stably-transfected cells, typically by contacting the cells with a senescence-inducing concentration of doxorubicin or other cytotoxic agent. These experiments are used to establish levels of reporter gene induction in the absence of a test compound.

The promoter-reporter constructs are tested for inducibility by doxorubicin under conditions that activate the corresponding genes. The best-regulated promoter constructs are used to develop stably transfected cell lines, and cell lines identified that have the strongest induction of the reporter under the conditions of drug treatment, as described in Example 4.

Experiments are also performed in the presence of a test compound in an identical manner as experiments performed in the absence of the test compound. Experiments are typically performed at a variety of concentrations of the test compound in cells induced with the same concentration of cytotoxic agent, and expression of the reporter gene determined and compared to reporter gene expression in cells induced with that concentration of cytotoxic agent in the absence of the test compound.

The results of these experiments identify test compounds that reduce, inhibit or prevent senescence-associated induction of disease-promoting senescence-associated genes in cells treated with a cytotoxic drug, and effective concentrations thereof. These results provide compounds useful for preventing induction of disease-promoting, particularly tumor cell growth-stimulating genes as a consequence of cytotoxic agent-induced senescence associated with conventional cancer treatments.

It should be understood that the foregoing disclosure emphasizes certain specific embodiments of the invention and that all modifications or alternatives equivalent thereto are within the spirit and scope of the invention as set forth in the appended claims.

TABLE 1
Genes downregulated in senescent relative to proliferating cell fractions in HCT116 cells separated after doxorubicin treatment
(genes confirmed by RT-PCR are shown in boldface)

| | Accession | Effects of^{a}: | | | |
|---|---|---|---|---|---|
| Gene Name | Number | p53 | p21 | Notes | B.D.E.^{b} |

Transcription factors and cofactors

| | | | | |
|---|---|---|---|---|
| HFH-11/Trident/Win/MPP2 | U74612 | ↓¹ | Positive cell growth regulator², | −3.3 |
| | | | downregulated in aging³ | |
| AND-1 | AJ006266 | | WD repeat, HMG-box | −2.4 |
| Structure specific recognition protein 1 (SSRP1) | M86737 | ↓¹ | Transcription elongation factor | −2.3 |
| Histone acetyltransferase 1 (HAT1) | AF030424 | ↓^{d} | Transcription cofactor | −2.1 |
| Zinc finger protein, Y-linked (ZFY) | M30607 | | Testis determination | −2.1 |

Mitosis/DNA segregation

| | | | | | |
|---|---|---|---|---|---|
| Ki-67 antigen | X65550 | | ↓^{d} | Chromatin condensation | −4.9 |
| XCAP-C condensin homolog | NM_005496 | | | Chromatin condensation | −4.2 |
| Centromere protein F (CENP-F) | NM_005196 | | ↓¹ | Kinetochore component, downregulated in | −3.8 |
| | | | | aging³ | |
| XCAP-H condensin homolog | D38553 | | ↓¹ | Chromatin condensation | −3.7 |
| BUBR1/BUB1B | AF053306 | | ↓¹ | Kinetochore, spindle checkpoint control | −3.6 |
| Kinesin-like DNA binding protein (Kid/Obp-2) | AB017430 | | ↓^{d} | Kinetochore | −3.1 |
| AIM-1/AIK-2 | NM_004217 | | ↓¹ | Centrosome regulator | −3.1 |
| Lamin B receptor | L25941 | ↓⁴ | | Nuclear envelope assembly | −3 |
| Apoptosis inhibitor 4 (survivin) | U75285 | | ↓^{d} | Centrosome; protects from mitosis- | −2.9 |
| | | | | associated apoptosis | |
| CDC2 | X05360 | ↓⁵ | ↓¹ | Mitosis initiation | −2.7 |
| CDC20 | AW411344 | | ↓^{d} | APC activation/anaphase onset, | −2.6 |
| | | | | downregulated in aging³ | |
| Mitotic kinesin-like protein-1 | H63163 | | | Spindle movement | −2.5 |
| Centromere protein E (CENP-E) | Z15005 | | ↓^{d} | Kinetochore | −2.5 |
| ZW10 interactor (hZwint-1/MPP5) | AW409765 | | ↓¹ | Kinetochore | −2.5 |
| Thyroid hormone receptor interactor 13 | AA134541 | | ↓¹ | homolog of a yeast pachytene checkpoint | −2.4 |
| (TRIP13)/HPV16 E1 binding protein | | | | protein | |
| Breast cancer 1 (BRCA1) | L78833 | ↓⁶ | | Centrosome duplication regulator, tumor | −2.3 |
| | | | | suppressor | |
| Homolog of rough deal (Rod) protein of | AF070553 | | | Chromosome segregation | −2.3 |
| Drosophila | | | | | |
| AIK-1/AIM-2/STK15 | NM_003600 | | ↓¹ | Centrosome regulator, protooncogene | −2.1 |
| | | | | amplified in cancers⁷ | |
| MAD2 | NM_002358 | ↓⁴ | ↓¹ | Kinetochore, spindle checkpoint control | −2.1 |
| Topoisomerase IIα | AF071747 | ↓⁴ | ↓¹ | DNA and chromosome segregation | −2.1 |
| Lamin B2 | M94363 | | ↓¹ | Nuclear envelope assembly | −2.1 |
| Pericentrin | AI970199 | | | Centrosome | −2 |
| Thymopoietin | U18271 | | ↓¹ | Nuclear envelope assembly | −2 |
| FK506-binding protein 5 | U71321 | | | Homologous to rodent TP2 involved in | −2 |
| | | | | testis-specific chromatin condensation | |
| Polo-like kinase (PLK1) | U01038 | | ↓¹ | Controls initiation and several other stages of | ND^{e} |
| | | | | mitosis, downregulated in aging³ | |

DNA replication/chromatin assembly

| | | | | | |
|---|---|---|---|---|---|
| Ribonucleotide reductase M1 (RRM1) | NM_001033 | | ↓¹ | Nucleotide synthesis | −3.4 |
| High-mobility group protein 1 (HMG1) | AW160834 | | ↓¹ | Chromatin component | −3.4 |
| Thymidine kinase 1 | NM_003258 | | ↓¹ | Nucleotide synthesis | −3.3 |
| MCM7/CDC47 | D55716 | ↓⁴ | ↓¹ | Replication licensing factor component | −3.3 |
| Thymidylate synthase | NM_001071 | | ↓¹ | Nucleotide synthesis, downregulated in | −3.2 |
| | | | | aging³ | |
| MCM2 (mitotin) | AW264268 | | ↓^{d} | Replication licensing factor component | −2.8 |
| Replication factor C (activator 1) (36.5kD) | AI651635, | | ↓¹ | PCNA clamp formation | −2.7, |
| | AW651734 | | | | 2.4^{c} |
| High-mobility group protein 2 (HMG2) | X62534 | ↓⁴ | ↓¹ | Chromatin component, downregulated in | −2.5 |
| | | | | aging³ | |
| Replication protein A3 (14kD) | NM_002947 | | ↓^{d} | Single-stranded DNA binding protein, | −2.1 |
| | | | | involved in replication and repair | |
| Gamma-glutamyl hydrolase | NM_003878 | | | Folate metabolism regulator | −2 |
| (folylpolygammaglutamyl hydrolase) | | | | | |
| MCM3 | NM_002388 | | ↓^{d} | Replication factor | −2 |

DNA repair

| | | | | |
|---|---|---|---|---|
| HEX1 (RAD2 homolog) | AF042282 | ↓¹ | Exonuclease | −3.7 |
| Flap endonuclease 1 (FEN1, RAD2 homolog) | AW246270 | ↓^{d} | Exonuclease, downregulated in aging³ | −3 |
| RAD51 homolog | D14134 | ↓^{d} | Similar to E. coli RecA | −2.4 |
| T(12;16) malignant liposarcoma fusion (TLS/FUS) | S62140 | ↓^{d} | Retinoid-inhibited, protooncogene⁸ | −2.2 |

RNA processing/trafficking

| | | | | |
|---|---|---|---|---|
| Heterogeneous nuclear ribonucleoprotein H1 | NM_005520 | ↓^{d} | | −2.1 |
| Acidic protein rich in leucines (APRIL) | Y07570 | ↓^{d} | RNA stability | −2.1 |
| Pre-mRNA cleavage factor Im (25kD) | AA738354 | ↓^{d} | | −2 |
| Heterogeneous nuclear ribonucleoprotein G | Z23064 | | | −2 |
| Heterogeneous nuclear ribonucleoprotein A2/B1 | NM_002137 | ↓^{d} | | −2 |
| Heterogeneous nuclear ribonucleoprotein A1 | AA173135 | ↓^{d} | | −2 |

Proliferation-associated

| | | | | |
|---|---|---|---|---|
| Insulin induced gene 1 (INSIG1/CL-6) | AW663903 | | Liver regeneration | −2.3 |
| Hyaluronan-mediated motility receptor (RHAMM) | U29343 | ↓¹ | Cell motility, oncogenic activity⁹ | −2.1 |
| FSH primary response (LRPR1) | NM_006733 | | FSH proliferative response | −2.1 |
| Six-transmembrane epithetial protein of the | AC004969 | | Overexpressed in carcinomas, potential | −2.1 |
| prostate (STEAP) | | | membrane transporter¹⁰ | |

Other

| | | | | | |
|---|---|---|---|---|---|
| Rabkinesin-6 | NM_005733 | | ↓^{d} | Golgi, intracellular transport | −3 |
| Vaccinia related kinase 1 | AA312869 | | ↓^{d} | p53 phosphorylation, possible Mdm-2 | −3 |
| | | | | interference | |
| Protein kinase C, theta | L07032 | | ↓^{d} | Signal transduction | −2.4 |
| Ubiquitin carrier protein | AI571293 | | | Proteolysis, downregulated in aging³ | −2.2 |
| Actin, γ1 | NM_001614 | | | | −2.1 |
| KIAA0008 | D13633 | | ↓¹ | | −4.6 |
| KIAA0101 | D14657 | ↓⁴ | ↓¹ | | −4 |
| KIAA0056 | AF070553 | | | | −2.3 |
| KIAA0225 | D86978 | | | | −2.1 |

^{a}Known changes in gene expression upon ectopic overexpression of p53 or p21
^{b}B.D.E., balanced differential expression (from Incyte UniGem V 2.0 hybridization analysis), in almost all cases underestimates the actual fold difference observed by RT-PCR
^{c}Two clones in the array were found to be derived from the same gene, the B.D.E. values for both clones are shown
^{d}effects of p21 induction in HT1080 fibrosarcoma cells, as determined by microarray hybridization (our unpublished data, not included in the original report¹).
^{e}not detected microarray hybridization but identified by RT-PCR

Reference List

1. B. D. Chang et al., Proc. Natl Acad. Sci. USA 97, 4291-4296 (2000).

2. H. Ye, A. X. Holterman, K. W. Yoo, R. R. Franks, R. H. Costa, Mol. Cell Biol. 19, 8570-8580 (1999).

3. D. H. Ly, D. J. Lockhart, R. A. Lerner, P. G. Schultz, Science 287, 2486-2492 (2000).

4. R. Zhao et al., Genes Dev. 14, 981-993 (2000).

5. K. Kannan, N. Amariglio, G. Rechavi, D. Givol, FEBS Lett. 470, 77-82 (2000).

6. P. Arizti et al., Mol. Cell Biol. 20, 7450-7459 (2000).

7. H. Zhou et al., Nat. Genet. 20, 189-193 (1998).

8. D. Perrotti et al., EMBO J. 17, 4442-4455 (1998).

9. C. L. Hall et al., Cell 82, 19-26 (1995).

10. R. S. Hubert et al., Proc Natl. Acad Sci. U.S.A 96, 14523-14528 (1999).

TABLE 2
Genes upregulated in senescent relative to proliferating cell fractions in HCT116 cells separated after doxorubicin treatment
(genes confirmed by RT-PCR are shown in boldface)

| | Accession | Effects of^{a}: | | | |
|---|---|---|---|---|---|
| Gene Name | Number | p53 | p21 | Notes | B.D.E.^{b} |

Table 2A
Transcription factors

| | | | | | |
|---|---|---|---|---|---|
| X-box binding protein 1 (XBP-1/HTF/TREB) | AW021229 | | | bZIP domain, c-Jun family, dimerizes with Fos¹ | 3.9 |
| Activating transcription factor 3 (ATF3) | N39944 | ↑² | | bZIP domain, dimerizes with c-Jun³ | 3.3 |
| C-JUN | AI078377 | | | AP-1, stress response⁴ | 2.5 |
| ELF-1 | AW503166 | | | ets domain factor, expressed in lymphoid and | 2.4 |
| | | | | epithelial tissues⁵ | |
| Ring finger protein 3 (RNF3) | AA403225 | | ↑^{d} | homolog of 73Ah regulator of Drosophila | 2.3 |
| Homolog of Drosophila muscleblind B protein | AF061261 | | | C3H-type zinc finger protein | 2.3 |
| (MBLL) | | | | | |
| SOX9/SRY (sex-determining region Y) | NM_000346 | | | HMG domain, retinoid-inducible⁶, involved in | 2.2 |
| | | | | chondrocyte differentiation⁷, | |
| Sjogren syndrome antigen A2 (60kD, | U44388 | | | Putative transcription regulator | 2.1 |
| ribonucleoprotein SS-A/Ro) | | | | | |
| Core promoter element binding protein | AL037865 | | | Kruppel-like family transcription factor, | 2 |
| (CPBP/ZF9/KLF8) | | | | activates keratin-4 promoter⁸ | |

Growth inhibitors, intracellular

| | | | | |
|---|---|---|---|---|
| Epithelial Protein Lost in Neoplasms (EPLIN) | AL048161 | | Decreased in multiple carcinomas¹¹ | 3.5 |
| B-cell translocation gene 1 (BTG1) | AI560266 | | Tumor suppressor¹² | 2.8 |
| B-cell translocation gene 2 (BTG2) | NM_006763 | ↑¹³ | Tumor suppressor¹³ | 2.1 |
| WIP1 | NM_003620 | ↑¹⁴ | p53-inducible protein phosphatase¹⁴ | 2 |

Growth inhibitors, secreted

| | | | | |
|---|---|---|---|---|
| Maspin | AA316156, | ↑¹⁵ | Serine protease inhibitor, downregulated in | 5.2, |
| | AI435384 | | neoplasms, inhibits tumor growth, metastasis, | 3.3^{c} |
| | | | angiogenesis¹⁶, upregulated in aging¹⁷ | |
| MIC-1 (Prostate differentiation factor, PTGF- | AB000584 | ↑¹⁸ | TGF-β family, downregulated in cancers, | 2.9 |
| β, PLAB) | | | induces growth arrest and apoptosis¹⁹ | |
| Insulin-like growth factor binding protein 6 | AA675888 | | Retinoid-inducible²⁰ | 2.7 |
| (IGFBP-6) | | | | |
| Amphiregulin | NM_001657 | | EGF/TGFα family secreted factor, promotes | 2.3 |
| | | | growth of normal epithelial cells but inhibits | |
| | | | carcinomas²¹, WT1-inducible²² | |

Other growth regulators

| | | | |
|---|---|---|---|
| CD44 antigen | X66733, | Adhesion molecule, growth modulator²³, | 3.9, |
| | X55150 | upregulated in aging¹⁷ | 2.1^{c} |
| Jagged-1 | U61276 | Notch ligand, stem cell growth, angiogenic | 2 |
| | | factor²⁴ | |

Cell adhesion and cell-cell contact

| | | | | |
|---|---|---|---|---|
| P-cadherin | NM_001793 | | Lost in prostate cancer³⁶ | 2.9 |
| Desmoplakin (DPI, DPII) | J05211 | | Decreased in neoplasms³⁷, upregulated in | 2.4 |
| | | | aging³⁸ | |
| PM5 protein (collagenase-related) | X57398 | | Homologous to cell adhesion proteins | 2.2 |
| CD63/ME491 antigen | X62654 | | | 2.1 |
| Mac-2 binding protein | X79089 | ↑²⁸ | ECM organizer³⁹ | 2 |
| Occludin | U53823 | | Tight junction protein | 2.1 |

ECM receptors

| | | | |
|---|---|---|---|
| Integrin β4 | X53587 | | 2.6 |
| Laminin, α3 (nicein/kalinin/BM600/epilegrin) | L34155 | | 2.4 |
| Syndecan 4 (amphiglycan, ryudocan) | D79206, | Involved in wound repair and angiogensis⁴⁰ | 2.3, |
| | NM_002999 | | 2.2^{c} |
| Integrin α6 | X53586 | | 2.2 |

Transmembrane signaling

| | | | |
|---|---|---|---|
| AHNAK nucleoprotein (desmoyokin) | M80899 | Activates PLC-γ⁴¹, decreased in | 2.1 |
| | | neuroblastomas⁴² | |
| CD24 antigen | AI745625 | Mucin-like glycoprotein, upregulated in breast | 2.1 |
| | | carcinoma⁴³ | |
| Lipocortin-2 (annexin A2) | W53011 | Substrate of src tyrosine kinase | 2 |

Ion transport and ion exchange

| | | | | |
|---|---|---|---|---|
| Phospholemman-like, 8kD (MAT-8) | AA826766 | | Chloride channel activator | 2.3 |
| Ferritin, heavy polypeptide 1 | AW575826 | ↑^{d} | Iron storage | 2.8 |
| Caveolin 2 | AI093287 | | Membrane compartmentalization | 2.2 |
| Neurogranin | Y09689 | ↑^{d} | Calmodulin binding protein, neural | 2.2 |
| H1 chloride channel | AI381979 | | Colocalizes with caveolin⁴⁴ | 2 |

Intracellular trafficking, cytoskeletal and scaffolding

| | | | |
|---|---|---|---|
| Interferon-induced protein 56 (IF1-56K/P56) | NM_001548 | Tetratricopeptide protein, Int6 interaction⁴⁵ | 3.2 |
| Major vault protein (lung resistance protein, | X79882 | Stress response, multidrug resistance | 2.4 |
| LRP) | | | |
| Macrophin (microfilament and actin filament | AB029290 | Cytoskeletal | 2.4 |
| cross-linker protein) | | | |
| Microtubule-associated protein 1B (MAP1B) | L06237 | Cytoskeletal, CK2 substrate | 2 |

Proapoptotic

| | | | | |
|---|---|---|---|---|
| NOXA | D90070 | ↑⁴⁶ | Bcl2 family member⁴⁶ | 2.7 |
| Fas antigen/APO-1 | M67454 | ↑⁴⁷ | Apoptotic signal receptor | 2.3 |

Keratins

| | | | | |
|---|---|---|---|---|
| Keratin 18 | X12881 | | Antiapoptotic⁴⁸ | 4 |
| Keratin 8 | X74929 | ↑⁴⁹ | Antiapoptotic⁴⁸ | 3.4 |
| Keratin 2A | AF019084 | | | 2.9 |
| Keratin 7 | M13955, | | | 2.6 |
| | AA307373 | | | 2.1^{c} |
| Keratin 15 | NM_002275 | | | 2.3 |
| Keratin 6B | L42611 | | | 2.1 |

Other

| | | | |
|---|---|---|---|
| High mobility group protein HMG2 homolog | AI191623 | | 5.4 |
| U1 small ribonucleoprotein 1SNRP homolog | AI400786 | | 3.7 |
| Retinaldehyde dehydrogenase 3 | U07919 | Retinoic acid synthesis | 3.2 |
| (ALDH6/RALDH3) | | | |
| Tumor differentially expressed 1 (TDE1) | NM_006811 | Transmembrane protein, homologous to mouse | 2.4 |
| | | gene increased in testicular tumors⁵⁰ | |
| Apolipoprotein E | K00396 | Alzheimer's, atherosclerosis | 2 |
| Incyte EST | X62654 | | 2.1 |
| 23815 human mRNA | U90916 | | 2.1 |

Table 2B
Growth regulators, intracellular

| | | | | | |
|---|---|---|---|---|---|
| p21 (Wafl/Cip1/Sdi1) | AA481712 | ↑⁹ | | Pleiotropic inhibitor of cyclin-CDK complexes, | 5.1 |
| | | | | inhibits or stimulates various transcription | |
| | | | | factors and cofactors¹⁰ | |
| Cyclin D1 (Bcl-1) | M73554, | ↑²⁵ | ↑²⁵ | G1/S transition; coregulated with p21 in | 2.8, |
| | X59798 | | | cancers²⁶ | 2.2^{c} |
| Serum-inducible kinase (Snk, polo-like) | NM_006622 | | | Putative cell growth regulator | 2.2 |

Mitogenic/antiapoptotic factors, secreted

| | | | | | |
|---|---|---|---|---|---|
| CYR61 | Y12084 | | | Mitogenic/angiogenic factor²⁷ | 3.3 |
| Prosaposin | J03015 | | ↑²⁸ | Antiapoptotic/mitogenic²⁹ upregulated in | 2.3 |
| | | | | aging³⁰ | |
| Transforming growth factor α (TGFα) | X70340 | ↑³¹ | | EGF-related mitogen³² | 2 |

Proteases

| | | | |
|---|---|---|---|
| Kallikrein 7 (serine protease 6) | L33404 | Upregulated in ovarian carcinoma³³ | 3.2 |
| Calpain-L2 | M23254 | | 2.3 |
| Neurosin (serine protease 9, Zyme, Protease | NM_002774 | Downregulated in breast cancers³⁴, upregulated | 2 |
| M) | | in ovarian carcinoma³⁵ | |
| Plasminogen activator, urokinase | D11143 | | 2 |

Other

| | | | | |
|---|---|---|---|---|
| Amyloid beta (A4) precursor protein (βAPP) | X06989 | ↑²⁸ | Alzheimer's disease amyloid precursor | 2 |
| Integral membrane protein 2B (BRI/ITM2B) | AW131784 | | Amyloid precursor in familial British | 2 |
| | | | dementia⁵¹ | |

^{a}Known changes in gene expression upon ectopic overexpression of p53 or p21
^{b}B.D.E., balanced differential expression (from Incyte UniGem V 2.0 hybridization analysis), in almost all cases underestimates the actual fold difference observed by RT-PCR
^{c}Two clones in the array were found to be derived from the same gene, the B.D.E. values for both clones are shown
^{d}effects of p21 induction in HT1080 firbrosarcoma cells, as determined by microarray hybridization (our unpublished data, not included in the original report²⁸).

Reference List

1. A. M. Reimold et al., Genes Dev. 14, 152-157 (2000).

2. T. Hai, C. D. Wolfgang, D. K. Marsee, A. E. Allen, U. Sivaprasad, Gene Expr. 7, 321-335 (1999).

3. C. D. Wolfgang, G. Liang, Y. Okamoto, A. E. Allen, T. Hai, J. Biol. Chem. 275, 16865-16870 (2000).

4. D. Bohmann et al., Science 238, 1386-1392 (1987).

5. A. G. Bassuk, K. P. Barton, R. T. Anandappa, M. M. Lu, J. M. Leiden, Mol. Med. 4, 392-401 (1998).

6. O. J. Marshall and V. R. Harley, Mol. Genet. Metab 71, 455-462 (2000).

7. W. Huang, U. I. Chung, H. M. Kronenberg, B. de Crombrugghe, Proc.Nat.Acad.Sci.U.S.A 98, 160-165 (2001).

8. J. Okano et al., FEBS Lett. 473, 95-100 (2000).

9. W. S. El Deiry et al., Cell 75, 817-825 (1993).

10. G. P. Dotto, Biochim.Biophys.Acta 1471, M43-M56 (2000).

11. R. S. Maul and D. D. Chang, Oncogene 18, 7838-7841 (1999).

12. J. P. Rouault et al., EMBO J. 11, 1663-1670 (1992).

13. J. P. Rouault et al., Nat. Genet. 14, 482-486 (1996).

14. M. Fiscella et al., Proc.Natl.Acad.Sci.U.S.A 94, 6048-6053 (1997).

15. Z. Zou et al., J.Biol.Chem. 275, 6051-6054 (2000).

16. F. E. Domann, J. C. Rice, M. J. Hendrix, B. W. Futscher, Int.J.Cancer 85, 805-810 (2000).

17. C. K. Lee, R. Weindruch, T. A. Prolla, Nat. Genet. 25, 294-297 (2000).

18. M. Tan, Y. Wang, K. Guan, Y. Sun, Proc.Natl.Acad.Sci. U.S.A 97, 109-114 (2000).

19. P. X. Li et al., J.Biol.Chem. 275, 20127-20135 (2000).

20. Y. P. Dailly, Y. Zhou, T. A. Linkhart, D. J. Baylink, D. D. Strong, Biochim.Biophys.Acta 1518, 145-151 (2001).

21. G. D. Plowman et al., Mol. Cell Biol. 10, 1969-1981 (1990).

22. S. B. Lee et al., Cell 98, 663-673 (1999).

23. P. Herrlich et al., Ann.N.Y.Acad.Sci. 910, 106-118 (2000).

24. L. Walker et al., Stem Cells 17, 162-171 (1999).

25. X. Chen, J. Bargonetti, C. Prives, Cancer Res. 55, 4257-4263 (1995).

26. J. S. de Jong, P. J. van Diest, R. J. Michalides, J. P. Baak, Mol.Pathol. 52, 78-83 (1999).

27. A. M. Babic, M. L. Kireeva, T. V. Kolesnikova, L. F. Lau, Proc.Natl.Acad.Sci. U.S.A 95, 6355-6360 (1998).

28. B. D. Chang et al., Proc.Natl.Acad.Sci.U.S.A 97, 4291-4296 (2000).

29. M. Hiraiwa, E. M. Taylor, W. M. Campana, S. J. Darin, J. S. O'Brien, Proc.Natl.Acad.Sci.U.S.A 94, 4778-4781 (1997).

30. P. P. Mathur et al., Biochem.Mol.Biol.Int. 34, 1063-1071 (1994).

31. T. H. Shin, A. J. Paterson, J. E. Kudlow, Mol.Cell Biol. 15,4694-4701 (1995).

32. V. Kumar, S. A. Bustin, I. A. McKay, Cell Biol.Int. 19, 373-388 (1995).

33. H. Tanimoto et al., Cancer 86, 2074-2082 (1999).

34. A. Anisowicz, G. Sotiropoulou, G. Stenman, S. C. Mok, R. Sager, Mol.Med. 2, 624-636 (1996).

35. H. Tanimoto, L. J. Underwood, K. Shigemasa, T. H. Parmley, T. J. O'Brien, Tumour.Biol. 22, 11-18 (2001).

36. D. F. Jarrard et al., Clin. Cancer Res. 3, 2121-2128 (1997).

37. A. Hiraki et al., Br.J. Cancer 73, 1491-1497 (1996).

38. D. H. Ly, D. J. Lockhart, R. A. Lerner, P. G. Schultz, Science 287,2486-2492 (2000).

39. T. Sasaki, C. Brakebusch, J. Engel, R. Timpl, EMBO J. 17, 1606-1613 (1998).

40. F. Echtermeyer et al., J.Clin.Invest 107, R9-R14 (2001).

41. F. Sekiya, Y. S. Bae, D. Y. Jhon, S. C. Hwang, S. G. Rhee, J.Biol.Chem. 274, 13900-13907 (1999).

42. E. Shtivelman, F. E. Cohen, J. M. Bishop, Proc.Natl.Acad.Sci. U.S.A 89, 5472-5476 (1992).

43. M. Fogel et al., Cancer Lett. 143, 87-94 (1999).

44. J. C. Edwards, Am.J.Physiol 276, F398-F408 (1999).

45. J. Guo and G. C. Sen, J. Virol. 74, 1892-1899 (2000).

46. E. Oda et al., Science 288, 1053-1058 (2000).

47. M. Muller et al., J.Exp.Med. 188, 2033-2045 (1998).

48. C. Caulin, C. F. Ware, T. M. Magin, R. G. Oshima, J.Cell Biol. 149, 17-22 (2000).

49. T. Mukhopadhyay and J. A. Roth, Anticancer Res. 16, 105-112 (1996).

50. M. Bossolasco, M. Lebel, N. Lemieux, A. M. Mes-Masson, Mol.Carcinog. 26, 189-200 (1999).

51. R. Vidal et al., Nature 399,776-781 (1999).

TABLE 3

PCR Amplification Primer Sequences

| | | SEQ ID | | SEQ ID |
|---|---|---|---|---|
| Gene | Sense (5′-3′) | NO | Antisense (5′-3′) | NO |
| AIK1 | TGGAATATGCACGACTTGGA | 1 | TTCTCTGAGCATTGGCCTGT | 63 |
| AIK2 (AIM1) | TGGGACACCCGACATCTTA | 2 | GCTCTTCTGCAGCTCGTTGTA | 64 |
| APRIL | TGCCCCAGCTTACCTACTTG | 3 | AATCCATGAGCAGTCCAAGC | 65 |
| BRCA1 | AAGAGAGAGCCCCAGAGTCA | 4 | GACCTTGGTGGTTTGTTCCA | 66 |
| BUBR1 | GAAGCGGAGCTATTGACCAG | 5 | GGGTGTGATAATGGGATCCT | 67 |
| CDC2 | AAGCCGGGATCTACGATACC | 6 | GGCCAAAATCAGCCAGTTTA | 68 |
| CDC20 | GAGGTGCAGCTATGGGATGT | 7 | TGTAATGGGGAGACCAGAGG | 69 |
| CDC47 | CGACAGGTGGTACAGGGTTT | 8 | CAGCCATCTTGTCGAACTCA | 70 |
| CENP-E | GTTGATCTTGCAGGCAGTGA | 9 | TCAGGAGCATCCGTGTTAAG | 71 |
| Condensin H | ACGACACCTCCAACTTTTGC | 10 | CCGCTAAGCATCTTGTCGTC | 72 |
| (XCAP-H) | | | | |
| GRCC8 | CAGGTGTTTTCCAAGGAGGA | 11 | GCTGTGAGTCCCAGTTTGGT | 73 |
| HEX1(RAD2) | ACTGCGTGGGATTGGATTAG | 12 | CTTGAATGGGCAGGCATAG | 74 |
| HFH-11B | TTCACAGCATCATCAGAGCA | 13 | TCGAAGGCTCCTGAACGTTA | 75 |
| (MPP2, Trident) | | | | |
| HMG1 | AGGGAGTTGTCAAGGCTGAA | 14 | CTGTGCCCAAACAAGAACCT | 76 |
| HPV16E1-BP | GACTCACAGCCCATCGATTT | 15 | CACCAGGGCGTCTTTATCAT | 77 |
| (TRIP13) | | | | |
| K167 | CAGACTCCATGTGCCTGAGA | 16 | CCCTGGAGAACATAGGCAAA | 78 |
| KIAA0008 | GCCAAGGGCAATGAAAACTA | 17 | ACCTGCTTTGCTGCTTGAGT | 79 |
| KIAA0101 | CTGAAGAGGCAGGAAGCAGT | 18 | TGGCACCATTGCAATAATcA | 80 |
| KIAA0166(rod) | GCAGCTCAAAGTCCACATCA | 19 | GGCCTTGCCCTCTTTAGAAT | 81 |
| MAD2 | TGGCCGAGTTCTTCTCATTC | 20 | CGCAGTTCCTCAGAATTGGT | 82 |
| Pericentrin | GAGCGAGGTCTCCATTTTGT | 21 | AGCTTCGTCTCCCAGCATAA | 83 |
| PLK1 | AAGAGATCCCGGAGGTCCTA | 22 | TCCCACACAGGGTCTTCTTC | 84 |
| Ribonucleotide | ACCAGCAAAGATGAGGTTGC | 23 | GCATCGGGGCAATAAGTAAA | 85 |
| reductase M1 | | | | |
| STEAP | GGCCTTCAGAACTTCAGCAC | 24 | GCTCAATCCAGGCATCTTCT | 86 |
| Survivin | GGACCACCGCATCTCTACAT | 25 | CTGGTGCCACTTTGAAGACA | 87 |
| TopoII a | AGGTGGTCGAAATGGCTATG | 26 | CACTTCCCACGTGTGGTTTAC | 88 |
| ZWint (MPP5) | GAGAACCAGTGGCAGCTACA | 27 | AATGATGGTTGGGAGGTGAG | 89 |
| Amphiregulin | CATTATGCTGCTGGATTGGA | 28 | TCATGGACTTTTCCGCACAG | 90 |
| APR (NOXA) | CCGGCAGAAACTTCTGAATC | 29 | GTGGTGAGTTGGCACTGAAA | 91 |
| ATF3 | GCTGGAATCAGTCACTGTcA | 30 | GCCTTCAGTTCAGCATTGAC | 92 |
| bAPP | GTCGTTCCTGACAAGTGCAA | 31 | TGTTCAGAGCACACCTGTGG | 93 |
| BRI | AGAAGAGCCTGGTGTTGGTG | 32 | GCAAATAGGTTCCAGCCTTG | 94 |
| BTG1 | CGGTGTCCTTCATCTCGAAG | 33 | TCGATAATCCATCCCCAAGA | 95 |
| BTG2 | AACAGGCCAGCACATACCTC | 34 | CTCTGCCGAGGACGTCATTA | 96 |
| Calpain L2 | GCAGGGATCTTTCACTTCCA | 35 | AGCTTGGGCAGTTGTCATTC | 97 |
| CD44 | GTGCCGGTTTGGAGGTGTAT | 36 | TAGGAGGGATTCTGTCTGTG | 98 |
| C-JUN | ATGAGGAACCGCATCGCTGGCT | 37 | GACCAAGTCCTTCCCACTCGTG | 99 |
| Cyclin D1 | AGGTCTGGGAGGAACAGAAG | 38 | AGCGTGTGAGGCGGTAGTAG | 100 |
| CYR61 | GAAAGTTTCCAGCCCAACTG | 39 | TACACTGGCTGTCCACAAGG | 101 |
| ELF-1 | TGTGGATCTAAGGGGAATGC | 40 | TCTTGCACCTGCTGTGTTTC | 102 |
| EPLIN b | AGAAAGGGGACCCTGACTGT | 41 | AAGATCCTCACCGTCCTTGA | 103 |
| FAS (APO-1) | ATTGCTCAAGAACCATGCTG | 42 | GTTGCTGGTGAGTGTGCATT | 104 |
| IGFBP-6 | AACCGCAGAGACCAACAGAG | 43 | GACCCCAAGCACAGCTTTAT | 105 |
| Integrin b4 | GTGACTGTCCCCTCAGCAAT | 44 | CAGCAGGCACAGTACTTCCA | 106 |
| Jagged-1 | TGGCTCTGTGAGACcAAcTG | 45 | TCACAATTCTGACCCATCCA | 107 |
| Keratin 18 | CAGCATGAGCTTCACGACTC | 46 | CTCCTTCTCGTTCTGGATGC | 108 |
| LRP | AGATCATTCAGGCCACCATC | 47 | CCGACAGCACATACACATCC | 109 |
| MAC2-BP | ACCATGAGTGTGGATGCTGA | 48 | ACAGGGACAGGTTGAACTGC | 110 |
| MASPIN | CCCTATGCAAAGGAATTGGA | 49 | GAAGCCTGTGGACTCATCCT | 111 |
| MBLL | TCCTGTTCCTTGGATTGGAC | 50 | AAAGTGGGCACTGGATGAAG | 112 |
| MIC-1 | CGGATACTCACGCCAGAAGT | 51 | CACATGGTCACTTGCACCTC | 113 |
| p21WAF | GGAAGACCATGTGGACCTGT | 52 | ATGCCCAGCACTCTTAGGAA | 114 |
| P-cadherin | GTGACAGCCACAGATGAGGA | 53 | TTTGGCCTCAAAATCCAAAC | 115 |
| Prosaposin | CCAGAGCTGGACATGACTGA | 54 | GTCACCTCCTTGACCAGGAA | 116 |
| PRSS6 | ATGGCAAGATCCCTTCTCCT | 55 | GGTCAGAGGGAAAGGTGACA | 117 |
| (Kallikrein 7) | | | | |
| PRSS9 | GGGGTCCTTATCCATCCACT | 56 | GGGATGTTACCCCATGACAC | 118 |
| (Neurosin) | | | | |
| RNF3 | AGAGATCAAGGGGGAGACCT | 57 | CACCGAGAGGCAATGTTCTT | 119 |
| SOX-9 | GGTTGTTGGAGCTTTCCTCA | 58 | TAGCCTCCCTCACTCCAAGA | 120 |
| Syndecan 4 | TCGATCCGAGAGACTGAGGT | 59 | GGTTTCTTGCCCAGGTCATA | 121 |
| TGFa | CAGGTCCGAAAACACTGTGA | 60 | AATTCTGTTGTGGGGAGGTG | 122 |
| WIP1 | CGACCTCGACTCACTCACAA | 61 | ATGGGGAAGGAGTGATCACA | 123 |
| XBP-1 | TAGCAGCTCAGACTGCCAGA | 62 | ACTGGGTGCAAGTTGTCCAG | 124 |

We claim:

1. A method for identifying a compound that induces senescence in a mammalian cell, the method comprising the steps of:
(a) culturing the mammalian cell in the presence and absence of the compound;
(b) assaying expression of at least one cellular gene in Table 2A in said cell in the presence of the compound with expression of said gene in the cell in the absence of the compound; and
(c) identifying compounds that induce senescence when expression of at least one cellular gene in Table 2A is higher in the presence of the compound than in the absence of the compound.

2. A method according to claim 1, wherein the mammalian cell is a p53 deficient cell.

3. A method according to claim 1, wherein the mammalian cell is a tumor cell.

4. The method of claim 1, where expression of the cellular gene of Table 2A is detected by hybridization to a complementary nucleic acid.

5. The method of claim 1, wherein expression of the cellular gene of Table 2A is detected using an immunological reagent.

6. The method of claim 1, wherein expression of the cellular gene of Table 2A is detected by assaying for an activity of the cellular gene product.

7. The method of claim 1, wherein the cellular gene is BTG1, BTG2, EPLIN, WIP1, Maspin,MIC-1, IGFBP-6 or amphiregulin.

8. A method according to claim 1, wherein induction of at least one of the cellular genes in Table 2A is assayed using a recombinant mammalian cell comprising a reporter gene operably linked to a promoter from a cellular gene in Table 2A and detecting increased expression of the reporter gene in the presence of the compound than in the absence of the compound.

9. A method according to claim 1, further comprising the steps of:
d) assaying expression of one or more genes in Table 2B; and
e) identifying compounds wherein expression of the genes in Table 2B is not greater in the presence of the compound than in the absence of the compound.

10. The method of claim 9, where expression of the cellular gene of Table 2B is detected by hybridization to a complementary nucleic acid.

11. The method of claim 9, wherein expression of the cellular gene of Table 2B is detected using an immunological reagent.

12. The method of claim 9, wherein expression of the cellular gene of Table 2B is detected by assaying for an activity of the cellular gene product.

13. A method for identifying a compound that induces senescence in a mammalian cell, the method comprising the steps of:
(a) culturing the mammalian cell in the presence and absence of the compound;
(b) assaying expression of at least one cellular gene in Table 2A in said cell in the presence of the compound with expression of said gene in the cell in the absence of the compound;
(c) assaying the recombinant mammalian cell for cell growth and morphological features of senescence; and
(d) identifying compounds that induce senescence when expression of at least one cellular gene in Table 2A is higher in the presence of the compound than in the absence of the compound and the cells are growth-inhibited and express morphological features of senescence in the presence of the compound.

14. A method according to claim 13, wherein the mammalian cell is a p53 deficient cell.

15. A method according to claim 13, wherein the mammalian cell is a tumor cell.

16. The method of claim 13, where expression of the cellular gene of Table 2A is detected by hybridization to a complementary nucleic acid.

17. The method of claim 13, wherein expression of the cellular gene of Table 2A is detected using an immunological reagent.

18. The method of claim 13, wherein expression of the cellular gene of Table 2A is detected by assaying for an activity of the cellular gene product.

19. The method of claim 13, wherein the cellular gene is BTG1, BTG2, EPLIN, WIP 1, Maspin, MIC-1, IGFBP-6 or amphiregulin.

20. A method according to claim 13, wherein induction of at least one of the cellular genes in Table 2A is assayed using a recombinant mammalian cell comprising a reporter gene operably linked to a promoter from a cellular gene in Table 2A and detecting increased expression of the reporter gene in the presence of the compound than in the absence of the compound.

21. A method according to claim 13 further comprising the steps of:
e) assaying expression of one or more genes in Table 2B; and
f) identifying compounds wherein expression of the genes in Table 2B is not greater in the presence of the compound than in the absence of the compound.

22. A method according to claim 20 further comprising the steps of:
f) assaying expression of one or more genes in Table 2B; and
f) identifying compounds wherein expression of the genes in Table 2B is not greater in the presence of the compound than in the absence of the compound.

23. The method of claims 21 or 22, where expression of the cellular gene of Table 2B is detected by hybridization to a complementary nucleic acid.

24. The method of claims 21 or 22, wherein expression of the cellular gene of Table 2B is detected using an immunological reagent.

25. The method of claims 21 or 22, wherein expression of the cellular gene of Table 2B is detected by assaying for an activity of the cellular gene product.

26. A method for identifying a compound that induces senescence in a mammalian cell, the method comprising the steps of:
(a) producing a recombinant mammalian cell by introducing into said mammalian cell a recombinant expression construct comprising a promoter from a cellular gene in Table 2A operably linked to a reporter gene;
(b) culturing the recombinant mammalian cell in the presence and absence of the compound;
(c) assaying expression of the reporter gene in said recombinant cell in the presence of the compound with expression of said reporter gene in the recombinant cell in the absence of the compound; and
(d) identifying compounds that induce senescence when gene expression of the reporter gene is higher in the presence of the compound than in the absence of the compound.

27. A method according to claim 26, wherein the mammalian cell is a p53 deficient cell.

28. A method according to claim 26, wherein the mammalian cell is a tumor cell.

29. The method of claim 26, wherein the promoter of the cellular gene is a promoter from BTG1, BTG2, EPLIN, WIP1, Maspin, MIC-1, IGFBP-6 or amphiregulin.

30. A method according to claim 26, further comprising the steps of:
e) assaying expression of one or more genes in Table 2B; and
f) identifying compounds wherein expression of the genes in Table 2B is not greater in the presence of the compound than in the absence of the compound.

31. The method of claim 30, where expression of the cellular gene of Table 2B is detected by hybridization to a complementary nucleic acid.

32. The method of claim 30, wherein expression of the cellular gene of Table 2B is detected using an immunological reagent.

33. The method of claim 30, wherein expression of the cellular gene of Table 2B is detected by assaying for an activity of the cellular gene product.

34. A method for identifying a compound that induces senescence in a mammalian cell, the method comprising the steps of:
(a) producing a recombinant mammalian cell by introducing into said mammalian cell a recombinant expression construct comprising a promoter from a cellular gene in Table 2A operably linked to a reporter gene;
(b) culturing the recombinant mammalian cell in the presence and absence of the compound;
(c) assaying expression of the reporter gene in said recombinant cell in the presence of the compound with expression of said reporter gene in the recombinant cell in the absence of the compound;
(d) assaying the recombinant mammalian cell for cell growth and morphological features of senescence; and
(e) identifying compounds that induce senescence when reporter gene expression is higher in the presence of the compound than in the absence of the compound and the cells are growth-inhibited and express morphological features of senescence in the presence of the compound.

35. A method according to claim 34, wherein the mammalian cell is a p53 deficient cell.

36. A method according to claim 34, wherein the mammalian cell is a tumor cell.

37. The method of claim 34, wherein the promoter of the cellular gene is a promoter from a BTG1, BTG2, EPLIN, WIP1, Maspin, MIC-1, IGFBP-6 or amphiregulin.

38. A method according to claim 34, further comprising the steps of:
f) assaying expression of one or more genes in Table 2B; and
g) identifying compounds wherein expression of the genes in Table 2B is not greater in the presence of the compound than in the absence of the compound.

39. The method of claim 38, where expression of the cellular gene of Table 2B is detected by hybridization to a complementary nucleic acid.

40. The method of claim 38, wherein expression of the cellular gene of Table 2B is detected using an immunological reagent.

41. The method of claim 38, wherein expression of the cellular gene of Table 2B is detected by assaying for an activity of the cellular gene product.

42. A method for identifying a compound that induces senescence in a mammalian cell, the method comprising the steps of:
(a) culturing the mammalian cell in the presence and absence of the compound;
(b) assaying expression of at least one cellular gene in Table 1 in said cell in the presence of the compound with expression of said gene in the cell in the absence of the compound; and
(c) identifying compounds that induce senescence when expression of at least one cellular gene in Table 1 is lower in the presence of the compound than in the absence of the compound.

43. A method according to claim 42, wherein the mammalian cell is a p53 deficient cell.

44. A method according to claim 42, wherein the mammalian cell is a tumor cell.

45. The method of claim 42, where expression of the cellular gene of Table 1 is detected by hybridization to a complementary nucleic acid.

46. The method of claim 42, wherein expression of the cellular gene of Table 1 is detected using an immunological reagent.

47. The method of claim 42, wherein expression of the cellular gene of Table 1 is detected by assaying for an activity of the cellular gene product.

48. The method of claim 42, wherein the cellular gene is HFH-11, STEAP, RHAMM, INSIG1, LRPR1.

49. A method according to claim 42, wherein inhibition of at least one of the cellular genes in Table 1 is assayed using a recombinant mammalian cell comprising a reporter gene operably linked to a promoter from a cellular gene in Table 1 and detecting decreased expression of the reporter gene in the presence of the compound than in the absence of the compound.

50. A method according to claim 41, further comprising the steps of:
d) assaying expression of one or more genes in Table 2B; and
e) identifying compounds wherein expression of the genes in Table 2B is not greater in the presence of the compound than in the absence of the compound.

51. A method according to claim 48, further comprising the steps of:
d) assaying expression of one or more genes in Table 2B; and
e) identifying compounds wherein expression of the genes in Table 2B is not greater in the presence of the compound than in the absence of the compound.

52. The method of claims 50 or 51, where expression of the cellular gene of Table 2B is detected by hybridization to a complementary nucleic acid.

53. The method of claims 50 or 51, wherein expression of the cellular gene of Table 2B is detected using an immunological reagent.

54. The method of claims 50 or 51, wherein expression of the cellular gene of Table 2B is detected by assaying for an activity of the cellular gene product.

55. A method for identifying a compound that induces senescence in a mammalian cell, the method comprising the steps of:
(a) culturing the mammalian cell in the presence and absence of the compound;
(b) assaying expression of at least one cellular gene in Table 1 in said cell in the presence of the compound with expression of said gene in the cell in the absence of the compound;
(c) assaying the recombinant mammalian cell for cell growth and morphological features of senescence; and
(d) identifying compounds that induce senescence when expression of at least one cellular gene in Table 1 is lower in the presence of the compound than in the absence of the compound and the cells are growth-inhibited and express morphological features of senescence in the presence of the compound.

56. A method according to claim 55, wherein the mammalian cell is a p53 deficient cell.

57. A method according to claim 55, wherein the mammalian cell is a tumor cell.

58. The method of claim 55, where expression of the cellular gene of Table 1 is detected by hybridization to a complementary nucleic acid.

59. The method of claim 55, wherein expression of the cellular gene of Table 1 is detected using an immunological reagent.

60. The method of claim 55, wherein expression of the cellular gene of Table 1 is detected by assaying for an activity of the cellular gene product.

61. The method of claim 55, wherein the cellular gene is HFH-11, STEAP, RHAMM, INSIG1, LRPR1.

62. A method according to claim 55, wherein inhibition of at least one of the cellular genes in Table 1 is assayed using a recombinant mammalian cell comprising a reporter gene operably linked to a promoter from a cellular gene in Table 1 and detecting decreased expression of the reporter gene in the presence of the compound than in the absence of the compound.

63. A method according to claim 55, further comprising the steps of:
e) assaying expression of one or more genes in Table 2B; and
f) identifying compounds wherein expression of the genes in Table 2B is not greater in the presence of the compound than in the absence of the compound.

64. A method according to claim 62, further comprising the steps of:
f) assaying expression of one or more genes in Table 2B; and
f) identifying compounds wherein expression of the genes in Table 2B is not greater in the presence of the compound than in the absence of the compound.

65. The method of claims 63 or 64, where expression of the cellular gene of Table 2B is detected by hybridization to a complementary nucleic acid.

66. The method of claims 63 or 64, wherein expression of the cellular gene of Table 2B is detected using-an immunological reagent.

67. The method of claims 63 or 64, wherein expression of the cellular gene of Table 2B is detected by assaying for an activity of the cellular gene product.

68. A method for identifying a compound that induces senescence in a mammalian cell, the method comprising the steps of:
(a) producing a recombinant mammalian cell by introducing into said mammalian cell a recombinant expression construct comprising a promoter from a cellular gene in Table 1 operably linked to a reporter gene;
(b) culturing the recombinant mammalian cell in the presence and absence of the compound;
(c) assaying expression of the reporter gene in said recombinant cell in the presence of the compound with expression of said reporter gene in the recombinant cell in the absence of the compound; and
(d) identifying compounds that induce senescence when expression of the reporter gene is lower in the presence of the compound than in the absence of the compound.

69. A method according to claim 68, wherein the mammalian cell is a p53 deficient cell.

70. A method according to claim 68, wherein the mammalian cell is a tumor cell.

71. The method of claim 68, wherein the promoter of the cellular gene is a promoter from HFH-11, STEAP, RHAMM, INSIG1, LRPR1.

72. A method according to claim 68, further comprising the steps of:
e) assaying expression of one or more genes Table 2B; and
f) identifying compounds wherein expression of the genes in Table 2B is not greater in the presence of the compound than in the absence of the compound.

73. The method of claim 72, where expression of the cellular gene of Table 2B is detected by hybridization to a complementary nucleic acid.

74. The method of claim 72, wherein expression of the cellular gene of Table 2B is detected using an immunological reagent.

75. The method of claim 72, wherein expression of the cellular gene of Table 2B is detected by assaying for an activity of the cellular gene product.

76. A method for identifying a compound that induces senescence in a mammalian cell, the method comprising the steps of:
(a) producing a recombinant mammalian cell by introducing into said mammalian cell a recombinant expression construct comprising a promoter from a cellular gene in Table 1 operably linked to a reporter gene;
(b) culturing the recombinant mammalian cell in the presence and absence of the compound;
(c) assaying expression of the reporter gene in said recombinant cell in the presence of the compound with expression of said reporter gene in the recombinant cell in the absence of the compound;
(d) assaying the recombinant mammalian cell for cell growth and morphological features of senescence; and
(e) identifying compounds that induce senescence when reporter gene expression is lower in the presence of the compound than in the absence of the compound and the cells are growth-inhibited and express morphological features of senescence in the presence of the compound.

77. A method according to claim 76, wherein the mammalian cell is a p53 deficient cell.

78. A method according to claim 76, wherein the mammalian cell is a tumor cell.

79. The method of claim 76, wherein the promoter of the cellular gene is a promoter from HFH-11, STEAP, RHAMM, INSIG1, LRPR1.

80. A method according to claim 76, further comprising the steps of:
g) assaying expression of one or more genes in Table 2B; and
g) identifying compounds wherein expression of the genes in Table 2B is not greater in the presence of the compound than in the absence of the compound.

81. The method of claim 80, where expression of the cellular gene of Table 2B is detected by hybridization to a complementary nucleic acid.

82. The method of claim 80, wherein expression of the cellular gene of Table 2B is detected using an immunological reagent.

83. The method of claim 80, wherein expression of the cellular gene of Table 2B is detected by assaying for an activity of the cellular gene product.

84. A compound that induces senescence in a mammalian cell wherein the compound is identified according to a method of claim 9, 21, 22, 30, 38, 50, 51, 63, 64, 72 or 80.

85. A compound according to claim 84 that is a non-retinoid compound.

86. A method for assessing efficacy of a treatment of a disease or condition relating to abnormal cell proliferation or neoplastic cell growth, the method comprising the steps of:
(a) obtaining a biological sample comprising cells from an animal having a disease or condition relating to abnormal cell proliferation or neoplastic cell growth before treatment and after treatment;
(b) comparing expression of at least one gene in Table 1, 2A or 2B after treatment with expression of said genes before treatment; and
(c) determining that said treatment has efficacy for treating the disease or condition relating to abnormal cell proliferation or neoplastic cell growth if expression of at least one gene in Table 2A and 2B is higher after treatment than before treatment or expression of at least one gene in Table 1 is lower after treatment than before treatment.

87. The method of claim 86, wherein the biological sample comprises tumor cells.

88. The method of claim 86, wherein the gene is a cellular gene in Table 2A.

89. The method of claim 88, wherein at least one cellular gene is BTG1, BTG2, EPLIN, WIP1, Maspin, MIC-1, IGFBP-6 or amphiregulin.

90. The method of claim 86, wherein the gene is a cellular gene in Table 1.

91. The method of claim 90, wherein the cellular gene is HFH-11, STEAP, RHAMM, INSIG1, LRPR1.

92. The method of claim 86, where expression of the cellular gene of Tables 1, 2A or 2B is detected by hybridization to a complementary nucleic acid.

93. The method of claim 86, wherein expression of the cellular gene of Tables 1, 2A or 2B is detected using an immunological reagent.

94. The method of claim 86, wherein expression of the cellular gene of Tables 1, 2A or 2B is detected by assaying for an activity of the cellular gene product.

95. A method for treating a disease or condition relating to abnormal cell proliferation or neoplastic cell growth, the method comprising the steps of administering to an animal having said disease or condition a therapeutically effective amount of a compound produced according to the method of claims 9, 21, 22, 30, 38, 50, 51, 63, 64, 72 or 80 that induces senescence in abnormally proliferating or neoplastic cells.

96. The method of claim 95 wherein the compound is a non-retinoid compound.

97. A method for identifying a compound that inhibits senescence-associated induction of cellular gene expression, the method comprising the steps of:
(a) contacting the cell with a cytotoxic agent at a concentration of said agent that inhibits cell growth;
(b) assaying the cell in the presence and absence of the compound for changes in expression of cellular genes induced when cells become senescent; and
(c) identifying the compound as an inhibitor of senescence-associated induction of cellular gene expression if expression of the cellular genes of subpart (b) is induced in the absence of the compound but is not induced in the presence of the compound.

98. The method of claim 97, wherein the cellular gene is cyclin D1, serum-inducible kinase, CYR61, prosaposin, transforming growth factor α □(TGFα), kallikrein 7, calpain-L2, neurosin, plasminogen activator urokinase, amyloid beta (A4) precursor protein (βAPP), or integral membrane protein 2B (BRI/ITM2B).

99. The method of claim 97, where expression of the cellular gene is detected by hybridization to a complementary nucleic acid.

100. The method of claim 97, wherein expression of the cellular gene is detected using an immunological reagent.

101. The method of claim 97, wherein expression of the cellular gene is detected by assaying for an activity of the cellular gene product.

102. A method according to claim 97, wherein the mammalian cell is a p53 deficient cell.

103. A method according to claim 97, wherein the mammalian cell is a tumor cell.

104. A method for identifying a compound that inhibits senescence-associated induction of cellular gene expression, the method comprising the steps of:
(a) producing a recombinant mammalian cell by introducing into said mammalian cell a recombinant expression construct comprising a promoter from cyclin D1, serum-inducible kinase, CYR61, prosaposin, transforming growth factor α □(TGFα), kallikrein 7, calpain-L2, neurosin, plasminogen activator urokinase, amyloid beta (A4) precursor protein (βAPP), or integral membrane protein 2B (BRI/ITM2B) operably linked to a reporter gene;
(b) contacting the cell with a cytotoxic agent at a concentration of said agent that inhibits cell growth;
(c) assaying expression of the reporter gene in said recombinant cell in the presence of the compound with expression of said reporter gene in the recombinant cell in the absence of the compound
(d) identifying the compound as an inhibitor of senescence-associated induction of cellular gene expression if expression of the cellular genes of subpart (c) is induced in the absence of the compound but is not induced in the presence of the compound.

105. The method of claim 104, where expression of the cellular gene is detected by hybridization to a complementary nucleic acid.

106. The method of claim 104, wherein expression of the cellular gene is detected using an immunological reagent.

107. The method of claim 104, wherein expression of the cellular gene is detected by assaying for an activity of the cellular gene product.
